(19) United States
(12) Patent Application Publication
(10) Pub. No.: US 2024/0002772 A1
(43) Pub. Date: Jan. 4, 2024

(54) SYSTEMS AND METHODS FOR PRODUCING A BIOLOGIC MOLECULE WITH HYDROCYCLONE CELL RETENTION DEVICE

(71) Applicants: Boehringer Ingelheim International GmbH, Ingelheim am Rhein (DE); Gregory Walter HILLER, Ridgefield, CT (US); Anta Maria KUNDU, rIDGEFIELD, CT (US)

(72) Inventors: Gregory Walter HILLER, Wakefield, MA (US); Anita Maria KUNDU, Medford, MA (US)

(21) Appl. No.: 18/248,543

(22) PCT Filed: Oct. 12, 2021
(86) PCT No.: PCT/US2021/054481

§ 371 Date: Apr. 11, 2023

Related U.S. Application Data
(60) Provisional application No. 63/090,909, filed on Oct. 13, 2020.

(51) Int. Cl. C12M 1/26; C12M 1/00; C12M 1/06; C12N 5/071

(52) U.S. Cl. CPC C12M 33/10; C12M 23/58; C12M 29/18; C12N 5/0602; C12M 27/02

(57) ABSTRACT

Systems and methods for continuous production of at least one biological molecule of interest are provided. More specifically, the systems include (i) at least one culture bioreactor; (ii) at least one hydrocyclone; and (iii) at least one production bioreactor; wherein the culture bioreactor and the production bioreactor are linked by the hydrocyclone. The methods include the steps of (i) culturing a plurality of host cells capable of producing a biologic molecule of interest in at least one culture bioreactor; (ii) inoculating at least one production bioreactor with cells obtained from step (i); and (iii) culturing the cells in the production bioreactor.

### FIELD OF THE ART

Disclosed herein are systems and methods for producing a biologic molecule of interest, which systems and methods include at least one hydrocyclone serving as a cell retention device. In certain embodiments, the systems and methods provide a two-stage, linked perfusion bioreactor system for producing proteins, such as antibodies.

### BACKGROUND

Over the last decade, biologic drugs have grown in importance in the treatment of human disease. However, manufacturing processes for biologics differ greatly from the manufacturing processes for traditional small molecule drugs, i.e., biologics are typically produced by engineered cells or microorganisms versus synthesized through chemical reactions. The production of biologics is therefore typically more complex and expensive than the production of small molecules.

Continuous bioprocessing offers one way to improve the quality of biologics manufacturing and reduce cost. Bioprocessing may be continuous from end-to-end or continuous with respect to certain processes, e.g., cell culture. Continuous bioprocessing as it relates to upstream operations generally refers to perfusion technologies. A perfusion cell culture process involves the constant or semi-constant feeding of fresh media and the constant or semi-constant removal of spent media and product while retaining high numbers of viable cells.

Removing spent media while keeping cells in culture can be done in various ways including techniques based on filtration, sedimentation, or centrifugation. Cell retention devices known in the art include spin filters, inclined settlers, continuous centrifugation, membrane filtration devices, or ultrasonic separators. However, each of these is known to suffer certain limitations and/or complexities.

For example, nearly any cell retention device that uses a membrane (as do many that are currently in use at large scale) will eventually plug with cell debris. The high cell densities and low viabilities often associated with highly productive perfusion processes further accelerate membrane fouling. Additionally, as membranes plug with cell debris they also begin to effectively function as ultrafiltration devices, retaining the high molecular weight product proteins within the bioreactor in a not easily predictable or reproducible manner. This is a disadvantage because in a continuous perfusion bioreactor system it is advantageous to have the product of interest be continuously removed from the bioreactor in the cell-free harvest and delivered to the downstream operation in a consistent manner. Other problems with cell retention devices known in the art include overheating, low separation efficiencies and long residence times in the device.

As a result, conventional continuous perfusion cell culture systems usually have working volumes below 2,000 L, and if operated under conditions where productivity is highest (e.g. high viable cell densities and high perfusion rates), require frequent change out of the membrane-based cell retention device due to plugging and product retention. Additionally, cell retention devices capable of handling very large volumes of cell-free culture harvest can be quite complex (e.g. many moving parts), expensive, damaging to cells through excessive shear forces, and prone to failure. As the cell retention devices are typically external to the bioreactor, effective cleaning and sterilization can also be challenging at the large scale. Moreover, batch and fed-batch remain the most common type of mammalian cell culture, regardless of scale.

It is known that a perfusion N−1 (seed) bioreactor using a membrane based cell retention device linked to an about 5-fold to about 20-fold larger continuous-flow stirred-tank reactor (CSTR, or chemostat) can enhance productivity (see, e.g., U.S. Patent Publication No. 2019/0031997 to Boehringer Ingelheim International GmbH).

There remains a need for an alternative cell culture method or system that overcomes the limitations associated with the current conventional perfusion culture systems, particularly for large-scale bioprocessing.

### SUMMARY OF THE INVENTION

Disclosed herein are bioprocessing systems and methods and more particularly, systems and methods for producing a biologic molecule of interest (e.g., an antibody) which include at least one hydrocyclone serving as a cell retention device.

In a first aspect, disclosed herein is a system for continuous production of at least one biologic molecule of interest, which system comprises: (i) at least one culture bioreactor (for example, a N−1 bioreactor or a N−1 perfusion bioreactor); (ii) at least one hydrocyclone; and (iii) at least one production bioreactor (for example, a N bioreactor or a continuously stirred tank reactor (CSTR) production bioreactor); wherein the culture bioreactor and the production bioreactor are linked by the hydrocyclone that serves as a cell retention device for the culture bioreactor, wherein the hydrocyclone produces an underflow stream containing concentrated cell culture and a partially cell-free overflow stream, and wherein the underflow stream containing concentrated cell culture is returned to the culture bioreactor and the partially cell-free overflow stream is directed to the production bioreactor.

In one embodiment, the system can operate in a highly productive steady state for more than six weeks, more than eight weeks, more than three months, more than six months or more than a year. A “highly productive” steady state is considered to be a calculated steady-state volumetric productivity of at least about 0.80 g/L/day, or about 0.84 g/L/day.

In a particular embodiment, the system can operate in a highly productive steady state indefinitely by restarting the culture bioreactor with cells of a lower generational number.

In a second aspect, disclosed herein is a system for continuous production of at least one biological molecule of interest, which system comprises: (i) at least one continuous (e.g., perfusion) culture bioreactor (for example, an N−1 bioreactor or N−1 perfusion bioreactor); (ii) at least one hydrocyclone; (iii) at least one production bioreactor (for example, an N bioreactor or a continuously stirred tank reactor (CSTR) production bioreactor); and (iv) at least one downstream component (e.g, capture or purification components); wherein the culture bioreactor and the production bioreactor are linked by the hydrocyclone that serves as a cell retention device for the culture bioreactor, wherein the hydrocyclone produces an underflow stream containing concentrated cell culture and a partially cell-free overflow stream, and wherein the underflow stream containing concentrated cell culture is returned to the culture bioreactor and the partially cell-free overflow stream is directed to the production bioreactor.

In a third aspect, disclosed herein is a method for continuously producing a biologic molecule of interest (e.g., an antibody), comprising (i) culturing a plurality of host cells capable of producing a biologic molecule of interest (e.g. an antibody) in at least one culture bioreactor (for example, an N−1 bioreactor or N−1 perfusion bioreactor); (ii) inoculating one or more production bioreactors (for example, an N bioreactor or a continuously stirred tank reactor (CSTR) production bioreactor) with cells obtained from step (i); and (iii) culturing the cells in the at least one production bioreactor under conditions that allow production of the biologic molecule of interest (e.g., an antibody), wherein the culture bioreactor and the production bioreactor are linked by a hydrocyclone which serves as a cell retention device for the culture bioreactor, which culture bioreactor receives the underflow from the hydrocyclone while the production bioreactor receives the overflow, thereby inoculating the production bioreactor.

In one embodiment, the method further comprises (v) harvesting a portion of the cultured host cells from the production bioreactor. In certain embodiments, the step of harvesting a portion of the cultured host cells from the production bioreactor occurs continuously. In certain embodiments, the step of harvesting a portion of the cultured host cells from the production bioreactor occurs periodically, for example one time per day, two times per day, three times per day, or one time per two days. Optionally, one or more additional steps can be conducted to further process the biologic molecule of interest. In one embodiment, the one or more steps are selected from clarifying, capturing, purifying, polishing, formulating, packaging steps or a combination thereof. These one or more additional steps may be continuous or non-continuous in nature, thereby providing a hybrid or continuous end-to-end process.

Compared with systems and/or methods which utilize a membrane-containing host cell retention device rather than a hydrocyclone to provide the link between the culture bioreactor and the production bioreactor, the systems and/or methods disclosed herein offer one or more improved properties, particularly when applied to large-scale systems. For example, in systems that exceed 1000 L in working volume of the production bioreactor, the systems and/or methods described herein, which utilize a hydrocyclone to provide the link between the culture bioreactor and the production bioreactor large, may provide certain improvements including, increased total separation efficiency, decreased media usage and/or increased production of at least one biologic molecule of interest. In certain embodiments, the improved properties are present at high viable cell densities (VCD) and or high perfusion rates (PF). In certain embodiments where the systems are of very large scale, systems which utilize a membrane-containing host cell retention device rather than a hydrocyclone to provide the link between the culture bioreactor and the production bioreactor, may be hindered by insufficient membrane area, which would result in lower overall productivities when compared to a hydrocyclone linked bioreactor system.

Additional advantages of the subject technology will become readily apparent to those skilled in this art from the following drawings and the detailed description. The drawings and description are to be regarded as illustrative in nature, and not as restrictive.

### BRIEF DESCRIPTION OF FIGURES

FIG. 1 is a diagram of the fluid dynamics in an exemplary hydrocyclone. Culture enters the hydrocyclone swirl chamber through two tangential inlets at the top of the device. A centrifugal field creates a primary vortex which pushes the cells towards the wall of the swirl chamber while the conical taper guides the cells downward until they exit in the underflow stream at the bottom of the device. Simultaneously, partially cell free material reverses direction and enters into a secondary vortex which travels upward and exits through the overflow stream at the top of the device.

FIG. 2 is a schematic illustrating an exemplary bioreactor configuration. In this configuration, the N−1 perfusion bioreactor using a hydrocyclone as the cell retention device continuously supplies the partially cell free overflow stream to a second continuous-flow stirred tank reactor (CSTR) operating as the N stage production bioreactor. The N−1 perfusion bioreactor was maintained at constant volume and was fed with perfusion medium. A 600 series Watson Marlow peristaltic pump supplies N−1 culture into the hydrocyclone at 3 L/min to reach an operating pressure of 2.4 bar. The underflow stream containing concentrated cell culture was recirculated back to the N−1 bioreactor while the partially cell-free overflow stream was added to the production CSTR with a second peristaltic pump (500 series Watson Marlow) at a flow rate of 370 mL/min. Concentrated nutrient feeds were also added directly to the production CSTR. The production CSTR did not use a cell retention device and whole cell harvest was removed from the bioreactor to maintain the production CSTR at constant volume.

FIG. 3 is a drawing of an exemplary hydrocyclone used as a cell retention device for an exemplary N−1 perfusion bioreactor. The dimensions shown on the diagram are described herein.

FIG. 4a is a graph showing the viable cell density (VCD) and percent cell viability as measured by trypan blue exclusion for an exemplary N−1 perfusion bioreactor using the hydrocyclone as a cell retention device. The viable cell density is displayed as closed diamonds and corresponds to the left y-axis. Percent cell viability is displayed as open diamonds and corresponds to the right y-axis. Vertical dashed lines illustrate the bounds of a steady state.

FIG. 4b is a graph showing the viable cell density and percent cell viability as measured by trypan blue exclusion for an exemplary production CSTR bioreactor simulating a 1:5 volume ratio in the linked bioreactor experiment using the hydrocyclone. The viable cell density is displayed as closed squares and corresponds to the left y-axis. Percent cell viability is displayed as open squares and corresponds to the right y-axis. Vertical dashed lines denote the bounds of a steady state.

FIG. 4c is a graph showing the viable cell density and percent cell viability as measured by trypan blue exclusion for an exemplary production CSTR bioreactor simulating a 1:10 volume ratio in the linked bioreactor experiment using the hydrocyclone. The viable cell density is displayed as closed triangles and corresponds to the left y-axis. Percent cell viability is displayed as open triangles and corresponds to the right y-axis. Vertical dashed lines denote the bounds of a steady state.

FIG. 4d is a graph showing the viable cell density and percent cell viability as measured by trypan blue exclusion for an exemplary production CSTR bioreactor simulating a 1:20 volume ratio in the linked bioreactor experiment using the hydrocyclone. The viable cell density is displayed as closed circles and corresponds to the left y-axis. Percent cell viability is displayed as open circles and corresponds to the right y-axis. Vertical dashed lines denote the bounds of a steady state.

FIG. 5a is a graph showing the residual glucose (closed diamonds) and lactate (open diamonds) concentrations in the N−1 perfusion bioreactor used in Example 1.

FIG. 5b is a graph showing the residual glucose (closed squares) and lactate (open squares) concentrations in an exemplary production CSTR simulating a 1:5 volume ratio in the linked bioreactor system using the hydrocyclone. The vertical dashed line denotes the timepoint at which High-End pH Delivery of Glucose (HIPDOG) control was suspended.

FIG. 5c is a graph showing the residual glucose (closed triangles) and lactate (open triangles) concentrations in an exemplary production CSTR simulating a 1:10 volume ratio in the linked bioreactor system using the hydrocyclone. The vertical dashed line denotes the timepoint at which High-End pH Delivery of Glucose (HIPDOG) control was suspended.

FIG. 5d is a graph showing the residual glucose (closed circles) and lactate (open circles) concentrations in an exemplary production CSTR simulating a 1:20 volume ratio in the linked bioreactor system using the hydrocyclone. The vertical dashed line denotes the timepoint at which High-End pH Delivery of Glucose (HIPDOG) control was suspended.

FIG. 6 is a plot showing the total separation efficiency (closed diamonds) and reduced separation efficiency (open diamonds) achieved by an exemplary hydrocyclone used as a cell retention device in the N−1 perfusion bioreactor. The calculations are described in the experimental section of this text.

FIG. 7 is a graph showing the perfusion rate (closed diamonds) and calculated effective cell bleed from an exemplary hydrocyclone overflow stream (open diamonds) in the N−1 perfusion bioreactor. The perfusion rate is listed in reactor volumes per day (RV/day) and it is calculated by dividing the volume of perfusion medium added to the N−1 bioreactor per day by the working volume of the N−1 bioreactor. The calculated cell bleed is also listed in RV/day and it is calculated by multiplying the perfusion rate by the ratio of the overflow stream viable cell density to bioreactor viable cell density. The dashed vertical lines illustrate the bounds of the steady states.

FIG. 8 is a graph showing the dilution rates in the 1:5 production CSTR (closed squares), 1:10 production CSTR (closed triangles), and 1:20 production CSTR (closed circles). The dilution rate is listed in RV/day and it is calculated by dividing the volume of whole cell harvest removed from the bioreactor each day by the working volume of the bioreactor. The vertical dashed lines illustrate the bounds of the steady states.

FIG. 9 is a graph showing the total mass of antibody product produced per volume plotted against time at steady state conditions in an exemplary linked bioreactor system using a hydrocyclone. The N−1 perfusion as a standalone unit operation is shown as closed diamonds, the 1:5 production CSTR is shown as open squares, the 1:10 production CSTR is shown as open triangles, and the 1:20 production CSTR is shown as open circles. The details of this material balance calculation are described in the experimental section of this application. When the bioreactor was at near steady state conditions with respect to perfusion rate or dilution rate, viable cell density, and metabolites, a linear regression was performed on the total product produced in grams per liter (g/L) plotted against time. The slope of the resulting line is the steady state volumetric productivity in grams per liter per day (g/L/day). The two steady states are circled with dashed lines.

FIG. 10 is a graph of the antibody product concentration or titer in the bioreactor. The N−1 perfusion is shown as closed diamonds, the 1:5 production CSTR is shown as open squares, the 1:10 production CSTR is shown as open triangles, and the 1:20 production CSTR is shown as open circles.

FIG. 11 is a diagram illustrating an exemplary cascading CSTRs bioreactor configuration. In this configuration, the N−1 bioreactor was operated at a constant volume as a CSTR. Perfusion medium was added to the N−1 CSTR as a whole cell bleed was semi-continuously removed from the bioreactor and fed to an N stage production CSTR that is 2.5 to 10 times larger than the N−1 CSTR. This production CSTR was also fed directly with concentrated feeds while whole cell harvest was continuously removed from the bioreactor to maintain the production CSTR at constant volume. Neither the N−1 CSTR nor the production CSTR utilized a cell retention device in this configuration.

FIG. 12a is a graph showing the viable cell density (VCD) and percent cell viability as measured by trypan blue exclusion for an exemplary N−1 CSTR. The viable cell density is displayed as closed diamonds and corresponds to the left y-axis. Percent cell viability is displayed as open diamonds and corresponds to the right y-axis. Vertical dashed lines illustrate the bounds of a steady state.

FIG. 12b is a graph showing the viable cell density and percent cell viability as measured by trypan blue exclusion for an exemplary production CSTR bioreactor simulating a 1:2.5 volume ratio in the cascading CSTRs experiment. The viable cell density is displayed as closed squares and corresponds to the left y-axis. Percent cell viability is displayed as open squares and corresponds to the right y-axis. Vertical dashed lines illustrate the bounds of a steady state and the timepoint at which the production CSTR was disconnected from the N−1 CSTR and operated as a standalone production CSTR.

FIG. 12c is a graph showing the viable cell density and percent cell viability as measured by trypan blue exclusion for an exemplary production CSTR bioreactor simulating a 1:5 volume ratio in the cascading CSTRs experiment. The viable cell density is displayed as closed triangles and corresponds to the left y-axis. Percent cell viability is displayed as open triangles and corresponds to the right y-axis. Vertical dashed lines illustrate the bounds of a steady state and the timepoint at which the production CSTR was disconnected from the N−1 CSTR and operated as a standalone production CSTR.

FIG. 12d is a graph showing the viable cell density and percent cell viability as measured by trypan blue exclusion for an exemplary production CSTR bioreactor simulating a 1:10 volume ratio in the cascading CSTRs experiment. The viable cell density is displayed as closed circles and corresponds to the left y-axis. Percent cell viability is displayed as open circles and corresponds to the right y-axis. Vertical dashed lines illustrate the bounds of a steady state.

FIG. 13 shows the dilution rate in reactor volumes per day (RV/day) in the cascading CSTRs experiment of Example 1. The graph shows the N−1 CSTR (closed diamond), the 1:2.5 production CSTR (open squares), the 1:5 production CSTR (open triangles) and the 1:10 production CSTR (open circles). The dilution rate is calculated as the volume of whole cell harvest removed from the CSTR per day divided by the working volume of the bioreactor. The dashed vertical lines indicate the bounds of the steady states.

FIG. 14a is a graph showing the residual glucose (closed diamonds) and lactate (open diamonds) concentrations in the N−1 CSTR in the cascading CSTRs experiment of Example 1.

FIG. 14b is a graph showing the residual glucose (closed squares) and lactate (open squares) concentrations in an exemplary production CSTR simulating a 1:2.5 volume ratio in the cascading CSTRs process. The vertical dashed line indicates the timepoint at which High-End pH Delivery of Glucose (HIPDOG) control was suspended.

FIG. 14c is a graph showing the residual glucose (closed triangles) and lactate (open triangles) concentrations in an exemplary production CSTR simulating a 1:5 volume ratio in the cascading CSTRs process. The vertical dashed line indicates the timepoint at which High-End pH Delivery of Glucose (HIPDOG) control was suspended.

FIG. 14d is a graph showing the residual glucose (closed circles) and lactate (open circles) concentrations in an exemplary production CSTR simulating a 1:10 volume ratio in the cascading CSTRs process. The vertical dashed line indicates the timepoint at which High-End pH Delivery of Glucose (HIPDOG) control was suspended.

FIG. 15 is a graph showing the total mass of antibody product produced per volume plotted against time at steady state conditions in the cascading CSTRs experiment of Example 1. The N−1 CSTR as a standalone unit operation is shown as closed diamonds, the 1:2.5 production CSTR is shown as open squares, the 1:5 production CSTR is shown as open triangles, and the 1:10 production CSTR is shown as open circles. The details of this material balance are described in the experimental section of this application. When the bioreactor was at near steady state conditions with respect to dilution rate, viable cell density, and metabolites, a linear regression was performed on the total product produced in grams per liter (g/L) plotted against time. The slope of the resulting linear line is the steady state volumetric productivity in grams per liter per day (g/L/day).

FIG. 16 is a graph showing the antibody product concentration or titer in the bioreactors for the cascading CSTRs experiment of Example 1. The N−1 CSTR is shown as closed diamonds, the 1:2.5 CSTR is shown as open squares, the 1:5 CSTR is shown as open triangles, and the 1:10 CSTR is shown as open circles.

### DETAILED DESCRIPTION OF THE INVENTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

To facilitate an understanding of the present subject technology, a number of terms and phrases are defined below:

### Definitions

The grammatical articles “one”, “a”, “an”, and “the”, as used herein, are intended to include “at least one” or “one or more”, unless otherwise indicated. Thus, the articles are used herein to refer to one or more than one (i.e., to at least one) of the grammatical objects of the article. By way of example, “a component” means one or more components, and thus, possibly, more than one component is contemplated and may be employed or used in an implementation of the described embodiments.

The term “about” generally refers to a slight error in a measurement, often stated as a range of values that contain the true value within a certain confidence level (usually ±la for 68% C.I.). The term “about” may also be described as an integer and values of ±20% of the integer.

The term “antibody” as used herein broadly refers to a protein capable of recognizing and binding to a specific antigen. The term specifically includes monoclonal antibodies, polyclonal antibodies, dimers, multimers, multispecific antibodies (e.g. bispecific antibodies), antibody fragments (e.g., Fab fragment, F(ab′)₂, Fv fragment or Fc fragment from a cleaved antibody, a scFv-Fc fragment, a minibody, a diabody or a scFv) and double and single chain antibodies. The term also includes human antibodies, humanized antibodies, chimeric antibodies and antibodies specifically binding cancer antigen. Furthermore, the term includes genetically engineered derivatives of an antibody. Antibodies, fragments of antibodies and genetically engineered antibodies may be obtained by methods that are known in the art.

The term “biopolymer” refers to a nucleic acid polymer (e.g., DNA, RNA), a polypeptide, a protein or a virus particle, which can be native or biologically or synthetically modified, including fragments, multimers, aggregates, conjugates, fusion products etc.

The term “biological product” as used herein refers to as used herein refers to a molecule that is produced and can be isolated from cellular culture. The biological product may be a biopolymer, e.g., a nucleic acid, polypeptide (e.g., protein), antibody, carbohydrate or lipid. The biological product produced using the system and method disclosed herein may be purified by methods known in the art for the given product, formulated into a final commercially relevant composition of interest (e.g. a pharmaceutical composition), and packaged in a suitable container.

The term “bioreactor” as used herein refers to manufactured or engineered device which provides a biologically active environment, e.g., containing cells or cell-free enzymes transform raw materials into biological products (and in some cases, less desirable impurities). Within the bioreactor, the conditions (e.g., gas, flow rates, temperature, pH and agitation speed/circulation rate) are generally homogenous can be precisely controlled by manipulating the supply of nutrients to the cells and the removal of impurities. Bioreactors can be classified by mode of operation as batch, fed-batch or continuous (e.g. a continuous culture or continuous stirred-tank reactor (CSTR)). Commonly bioreactors are cylindrical or cubic, ranging in size from milliliters to cubic meters, and are often made of stainless steel or plastic. In certain embodiments, the bioreactor may be single-use or disposable. A “chemostat” is a bioreactor to which fresh medium is continuously added, while culture liquid is continuously removed to keep the culture volume constant.

The term “cell density” as used herein refers to that number of cells present in a given volume of medium.

The terms “cell retention system” or “cell retention device” as used herein refer to a means of selectively retaining viable cells within a bioreactor such that the density of cells in the fluid leaving the bioreactor is lower than the density of cells in the fluid within the bioreactor. In this sense, a cell retention device is different from a cell separation device.

The term “cell viability” as used herein refers to the ability of cells in culture to survive under a given set of culture conditions or experimental variations. The term as used herein also refers to that portion of cells which are alive at a particular time in relation to the total number of cells, living and dead, in the culture at that time.

The term “continuous” as used herein in the context of a unit (e.g., an individual bioreactor) refers to a unit that operates over a period of time, e.g., a period of days, weeks, months or years.

The term “continuous” in the context of a process refers to two or more integrated (physically connected) continuous unit operations with zero or minimal hold volume in between. If all the unit operations are continuous and integrated, such processes are also referred to as fully continuous or end-to-end continuous. A process is hybrid if it is composed of both batch and continuous unit operations, e.g., a continuous upstream process (cell culture and synthesis of the target protein) and a batch downstream (purification and formulation of the protein into a drug substance or drug product). In the specific context of linked bioreactors described herein, the term “continuous” refers to a constant or non-periodic liquid transfer.

Generally, upon initiation of the system, the rate of cell bleed/transfer from the culture bioreactor to the production bioreactor, either in continuous or semi-continuous mode will be lower than the rate of cell growth so that the cell density can increase in the perfusion reactor to reach a steady-state condition. Once at steady state the cell bleed rate will be identical, or very slightly lower than the growth rate. There will always be a low level of cell death occurring in the perfusion bioreactor. At steady state, the bleed rate of cells from the perfusion bioreactor will be slightly lower than the steady state growth rate, i.e. the growth rate minus the death rate equals the bleed rate.

The term “culture” and “cell culture” and “mammalian cell culture” as used herein refer to a cell population, either surface-attached or in suspension that is maintained or grown in medium under conditions suitable to survival and/or growth of the cell population. These terms can also refer to the cell population and the medium in which the population is suspended.

The term “culture bioreactor” (e.g., a N−1 bioreactor, a N−1 perfusion bioreactor) refers to a bioreactor upstream of a production reactor, which cultures cells (e.g. mammalian cells) to serve as a source of inoculum for the production reactor, i.e., serves as a seed bioreactor. The inoculum is bled/transferred to the production bioreactor as overflow from a hydrocyclone which serves as a cell retention device for the culture bioreactor.

The term “downstream components” with respect to bioprocessing or biomanufacturing includes any and all components downstream of the production bioreactor. These may include, for example, components to permit clarification, capture, purification, polishing, formulation and/or packaging. In certain embodiments, the components are chromatography columns. The term “downstream steps” has a similar meaning, with respect to production of the biomolecule of interest in the production bioreactor.

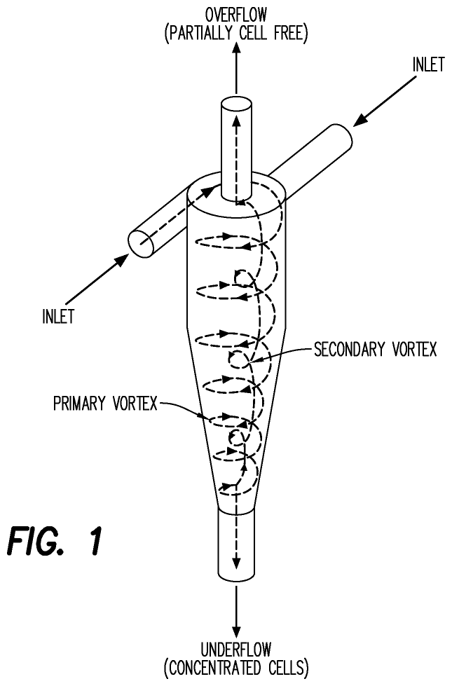

The term “hydrocyclone” in this context refers to a device which separates or sorts particles in a liquid suspension based on the ratio of their centripetal force to fluid resistance. A hydrocyclone will normally have a cylindrical section at the top where liquid is being fed tangentially, and a conical base. Various dimensions of the hydrocyclone, for example angle and length of the conical section, as well as other dimensions of the inner geometry of the hydrocyclone, can affect the operating characteristics of the device. Hydrocyclones generate an internal centrifugal force as fluid circulates through the device at high flowrates, separating cells from bulk culture fluid. Fluid enters through one or two small ports near the top of the device directing the inflow tangential to the inner conical shape. Flow through the chamber creates a primary vortex pushing cells to the wall while the conical taper guides the concentrated cells downward to leave through the bottom port (the underflow). Simultaneously, partially cell-depleted fluid moves upward creating a secondary vortex until it exits the device through a port at the top (the overflow) (FIG. 1).

The term “hybrid” as used herein with respect to a process refers to a process that includes both continuous and batch sub-processes. A hybrid process may include, for example, a continuous upstream process and a batch downstream process or a batch upstream process and a continuous downstream process.

The term “impurities” as used herein refers to refers to undesired chemical or biological compounds produced during the culturing process. Impurities may include e.g. ethyl alcohol, butyl alcohol, lactic acid, acetone ethanol, gaseous compounds, peptides, lipids, ammonia, aromatic compounds, and DNA and RNA fragments, as well as media components or break down products of the biological products.

The term “inoculum” as used herein refers to a partially cell-depleted fluid that is bled/transferred from the culture bioreactor to the production bioreactor to serve as a course of host cells for culture therein. The inoculum is the intermediate product of the hydrocyclone, which serves as a cell retention device for the culture bioreactor, providing the overflow to the production bioreactor as inoculum.

The term “isolated” as used herein refers to a biological molecule or group of similar molecules that have been subjected to fractionation to remove various other components and that retain substantially its expressed biological activity. Where the term “substantially purified” is used, this designation will refer to a composition in which the active form of the nutrients of the composition constitute about 10%, 20%, 30%, 40%, 50%, 60%, 70%, 80%, 90%, 95% or more of the total molecules in the composition.

The term “media” or “medium” or “cell culture medium” as used herein refers to a solution containing nutrients which nourish cultured cells. Typically, these solutions provide essential and non-essential amino acids, vitamins, energy sources, lipids, and trace elements required by the cell for minimal growth and/or survival. The solution can also contain components that enhance growth and/or survival above the minimal rate, including hormones and growth factors. The solution is formulated to a pH and salt concentration optimal for cell survival and proliferation.

The term “linked bioreactor” as used herein refers to two or more bioreactors having a direct or indirect connection (e.g., a hydrocyclone provides an indirect linkage). In embodiments described herein, the linked bioreactor may be a two-stage linked-bioreactor system, where the first stage is a culture or “seed” stage (bioreactor) and the second stage is a production stage (bioreactor).

The term “perfusion bioreactor” or “culture bioreactor” as used herein refers to a bioreactor that is continuously operated (e.g., an input stream and an output stream have a non-zero flow rate over a specified period of time) such that cells are retained within a reactor chamber of the bioreactor but at least a portion of the cell culture medium is continuously removed (and replenished). In certain embodiments disclosed herein, both the culture and the production bioreactor are perfusion bioreactors.

The term “perfusion culture” as used herein refers to a method of culturing cells in which fresh media is provided continuously or semi-continuously to the culture over time and previously introduced media (with impurities) are removed. The fraction of media removed and replaced each day can vary depending on the particular cells being cultured, the initial seeding density, and the cell density at a particular time. “RV or “reactor volume” means the volume of the culture medium present at the beginning of the culturing process (e.g., the total volume of the culture medium present after seeding). In certain embodiments, during the start-up of the production bioreactor, the working volume is about 70%, or about 60 to about 80%, of the final working volume. Typically, over the first few days (e.g. about 2 to about 4 days) after the start-up, the volume accumulates (or increases) in the bioreactor until reaching the final working volume. Dilution rates in RV/day are calculated based on final working volume, not the working volume starting at the beginning of culturing (i.e. at start-up).

The terms “production bioreactor” or “N bioreactor” or “CSTR bioreactor” or “CSTR production bioreactor” as used interchangeably herein refer to a bioreactor that does not utilize a cell retention system or device. The production bioreactor is linked to one or more upstream culture bioreactor(s) and receives inoculum from the culture bioreactor(s). The production bioreactor is uniformly mixed and has a fluid in-flow that is equivalent to the fluid out-flow, therefore, maintaining a near constant volume. Such a production bioreactor will often achieve, although not necessarily, a ‘chemically static’ or ‘steady-state’ environment when operating for sufficiently long periods of time, meaning that cell densities and other aspects of the culture (e.g., concentrations of nutrients, etc.) will reach a stable (i.e. steady or static) state, and is therefore also commonly referred to as a ‘chemostat’. The fluid in-flow to and/or out-flow from the production bioreactor may be independently continuous or semi-continuous. Such production bioreactor operates continuously for a period or equal or greater than 3, 4, 5, or 6 weeks.

The term “semi-continuous,” in the context of liquid transfer to and/or from a bioreactor, as used herein means ‘periodic’ or refers to a scenario in which liquid (e.g., media alone and/or with cells, cell bleed) is added to and/or removed from the bioreactor once every however long period of time. For example, once every 1, 2, 5, 10, 15, 30, 45 or 60 minutes, or once every hour, or once every 2-3 hours, or once every however long period of time from 1 minutes to 24 hours, a burst of liquid is transferred from and/or to the bioreactor for a period extending from few seconds (e.g., 1 sec., 2 sec., 5 sec. 10 sec., 20 sec. or 60 sec.) to several minutes (e.g. 2 min. 5 min., 10 min., 25 min., 50 min, 120 min. or 240 min.).

The term “working volume” refers to the portion of the total volume of a bioreactor. In some embodiments, the working volume is substantially constant even after feeding and/or sampling have occurred.

Hydrocyclone

Disclosed herein is a hydrocyclone for use in the systems and methods described herein. Generally, the hydrocyclone functions as a cell retention device in fluid communication with a culture bioreactor and a production bioreactor, wherein the hydrocyclone facilitates retention of viable cells within the culture bioreactor such that the density of cells in the fluid leaving the culture bioreactor is lower than the density of cells in the fluid within the culture bioreactor.

In certain embodiments, the hydrocyclone functions as, or replaces, a cell retention device. In certain embodiments, the hydrocyclone functions as, or replaces, a cell retention device in a system and method for production of a target biologic molecule (e.g., a biopolymer such as a protein). In certain embodiments, the hydrocyclone functions as, or replaces, a cell retention device in a system and method for production of a target biologic molecule in a large-scale (>1,000 L) production system.

Typically, conventional membrane-based cell retention systems in use today are 100% cell retention systems. When using a membrane-based cell retention system, a separate bleed of cells is taken directly from the N−1 bioreactor and transferred to the production bioreactor. At large scale, membrane-based cell retention systems are expensive, prone to pluging resulting in failure of the system, difficult to clean and reuse, difficult to sterilize, and engender additional risk of contamination to the process since they may need to be changed out during a continuous long-duration culture process. Replacing the membrane-based cell retention device with a hydrocyclone, as described herein, allows for such systems for the production of a target biologic molecule to be scaled up beyond that which feasible for systems relying on membrane-based cell retention devices.

In one embodiment, the hydrocyclone exhibits one or more improved properties relative to a comparable conventional membrane cell retention device, wherein the one or more improved properties are exhibited at high viable cell densities (VCD) and/or high perfusion rates (PR). High viable cell densities are in the range of about 20×10⁶ cells/mL to about 250×10⁶ cells/mL. High perfusion rates are at least about 1 VVD (volume of media per bioreactor volume per day).

In a particular embodiment, about 85 to about 95%, more particularly about 90 to about 95%, of the cells that enter the hydrocyclone are retained and returned to the culture bioreactor via the underflow (or underflow stream), more particularly, about 85%, about 87%, about 90%, about 92%, about 94% or about 95%.

Hydrocyclones are known in the art. In one embodiment, the hydrocyclone has a cylindrical section at the top where liquid enters and a conical base. Typically, the hydrocyclone has exits on the axis in opposing directions: the larger on the underflow (or accept) and a smaller at the overflow (or reject). The underflow stream is generally the denser or thicker fraction, while the overflow stream is the lighter or more fluid fraction. The overflow stream provides a partially cell-depleted inoculum to the production reactor.

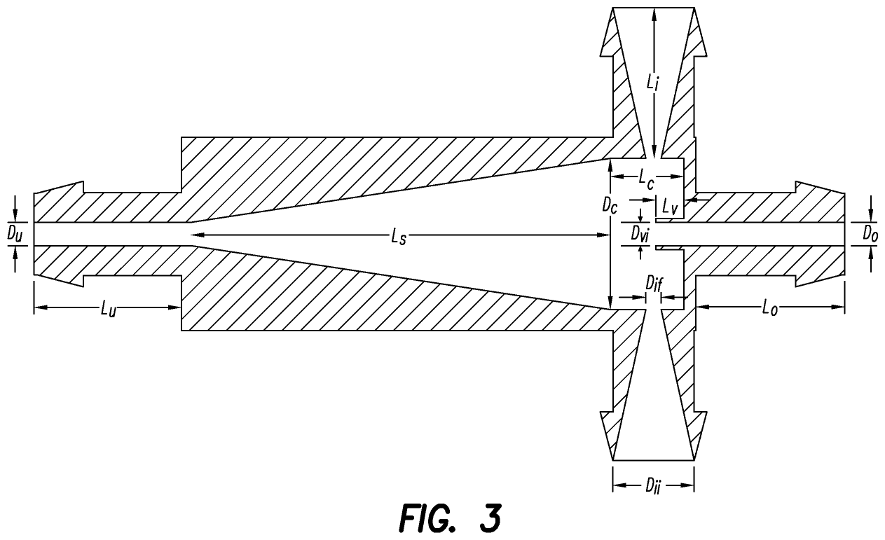

The geometry of the hydrocyclone may vary. In one embodiment, the hydrocyclone has the geometry shown in FIG. 3 and/or Table 1.

The dimensions of the hydrocyclone may vary. As referred to herein, dimensions of the hydrocyclone include, for example, overflow diameter (Dₒ), underflow diameter (Dᵤ), chamber diameter (D_{c}), conical section length (Lₛ), cylindrical section length (L_{c}), vortex finder length (Lᵥ), and underflow outlet length (Lᵤ), inlet initial diameter (Dᵢᵢ), inlet final diameter (Dᵢᵢ), vortex finder inner diameter (Dᵥᵢ), vortex finder wall thickness (W), overflow outlet length (Lₒ), and inlet length (Lᵢ). Generally, the vortex finder inner diameter is equal to the overflow diameter.

In one embodiment, the overflow diameter is in the range of about 1 to about 3 mm. In one embodiment, the underflow diameter is in the range about 1 to about 4 mm. In one embodiment, the chamber diameter is in the range about 5 to about 15 mm, or about 8 to about 12 mm. In one embodiment, the conical section length is in the range about 50 to about 170 mm, or about 60 to about 80 mm. In one embodiment, the cylindrical section length is in the range of about 2 to about 20 mm, or about 2 to about 6 mm. In one embodiment, the vortex finder length is in the range of about 1 to about 4 mm. In one embodiment, the length of the underflow outlet length is in the range of about 0 to about 40 mm, or about 1 to about 40 mm.

In one embodiment, the inlet initial diameter is in the range of about 3 to about 13 mm, or about 6 to about 11 mm. In one embodiment, the inlet final diameter is in the range of about 1 to about 3 mm. In one embodiment, the vortex finder inner diameter (Dᵥᵢ) is in the range of about 1 to about 3 mm. In one embodiment, the vortex finder wall thickness (W) is in the range of about 0.3 to about 2 mm, or about 0.3 to about 1 mm. In one embodiment, the, the overflow outlet length (Lₒ) is in the range of about 0 to about 40 mm, or about 1 to about 40 mm. In one embodiment, the inlet length (Lᵢ) is in the range of about 10 to 50 mm.

In a certain embodiment, the dimensions of each part of the hydrocyclone are proportional.

In a particular embodiment, the hydrocyclone has the configuration shown in FIG. 1. In a particular embodiment, the hydrocyclone has the configuration shown in FIG. 3. In a particular embodiment, the hydrocyclone has the dimensions shown in Table 1.

In one embodiment, the hydrocyclone is suitable use in systems and methods disclosed herein. In another embodiment, the hydrocyclone is a component of the systems and methods disclosed herein.

In certain embodiments, the hydrocyclone is linked to an upstream culture bioreactor. In one embodiment, the hydrocyclone is linked to the culture bioreactor by a peristaltic pump. A stream of concentrated cells is returned from the hydrocyclone to the culture reactor in an unrestricted underflow stream. Generally, a peristaltic pump (for example, a 600 series, Watson Marlow, Wilmington, MA) can be used to supply flow from the culture bioreactor through tubing (for example, a 9.5-mm inner diameter (ID) tubing) to the hydrocyclone. In certain embodiments, the flow from the culture reactor to the hydrocyclone is at a rate of about 2 to about 4 L/min, or about 3 L/min to create a desired pressure drop (for example a pressure drop of about 2.4-2.6 bar) within the device. In certain embodiments, the stream of concentrated cells returned to the culture bioreactor in the underflow stream is unrestricted. In certain embodiments, the underflow stream was returned to the culture reactor through a desired length (for example, about 15 cm) of tubing which fits over the lower portion of the hydrocyclone (for example, tubing with an inner diameter of 25.4-mm.) The tubing from the lower portion of the hydrocyclone may be connected to a narrower portion of tubing which connects to the culture bioreactor. For example, the 25.4 mm ID tubing can be reduced to 9.5 mm ID tubing over 4 cm using a 3D printed dual hose barb reducer. The 9.5 mm ID tubing can be attached to the reducer and welded onto the bioreactor.

Typically, a second peristaltic pump (for example a 500 series, Watson Marlow, Wilmington, MA) can be used to control the flow of the partially cell-free overflow stream leaving the culture bioreactor/hydrocyclone system through tubing (for example, a 9.5-mm inner diameter (ID) tubing) to the production reactor. In certain embodiments, the flow from the hydrocyclone to the production reactor is at a flow rate of about 320 to about 420 mL/min, or about 370 mL/min to achieve the desired maximum daily perfusion volume (for example a volume of about 532 L).

In certain embodiments, the hydrocyclone is linked to a downstream production bioreactor. In one embodiment, the hydrocyclone is linked to the downstream production bioreactor by a peristaltic pump.

The hydrocyclone may be suitable for use in a bench-sized system or method, a pilot-sized system or method, or a manufacturing-sized system or method, in each case for the culture of host cells and in particular, the production of at least one biologic molecule from such host cells, including but not limited to the systems and methods disclosed herein. In one embodiment, the hydrocyclone has the size and other appropriate dimensions for a bench-sized system, a pilot-sized system or a manufacturing-sized system.

Generally, for the systems and methods described herein, as the size or capacity of the system increases the number of hydrocyclones used in the system may be increased to provide the increased production of the system or method. For example, when the capacity or fluid volume of the system is increased or scaled-up, the number of hydrocyclones in the system may be increased to accommodate the increase in capacity or fluid volume. In certain embodiments, when two or more hydrocyclones are included in the system, said hydrocyclones are operated in parallel.

The hydrocyclone may be made of any suitable material. In certain embodiments, the hydrocyclone is made of metal (e.g., steel) or plastic (e.g., polypropylene or polyurethane). The hydrocyclone may be made by conventional manufacturing methods or additive manufacturing (i.e., 3D printing).

In certain embodiments, the hydrocyclone is made of metal. In certain embodiments, the hydrocyclones are constructed from stainless steel, which can be cleaned and sterilized in place on industrial bioreactors.

In certain embodiments, the hydrocyclones are made from plastics (e.g., biocompatible plastics by conventional or additive manufacturing (i.e., 3D printing). They can be sterilized by gamma irradiation or heat and provided as single use components.

In a particular embodiment, the hydrocyclone is made of 3D printed biocompatible plastic. Any suitable means of 3D printing may be used, including methods of material extrusion, vat polymerization (e.g. stereolithography (SLA)), powder bed fusion and powder bed jetting. In one embodiment, the hydrocyclone is 3D printed using an inverted stereolithography printer using an autoclavable and biocompatible resin.

In one embodiment, the hydrocyclone configuration is characterized by low energy consumption.

The hydrocyclones described herein can be used to separate cells, or at least reduce the concentration of cells, from a volume of about 200 to about 550 liters of cell culture fluid per day which would flow out of the overflow stream and into the CSTR. In certain embodiments, the volume of the inner flow chamber in the hydrocyclones is less than about 14 mL, less than about 10 mL, or less than about 5 mL. In certain embodiments, the volume of the inner flow chamber in the hydrocyclones is in the range of about 0.4 to about 14 mL, or about 2 mL to about 5 mL.

### System

The hydrocyclone disclosed herein may be utilized as a component of system suitable for use in culturing cells and, in certain embodiments, also producing one or more biologic molecules of interest.

Traditionally, systems for producing biologic products have been operated in batch or fed-batch modes of operation. In certain embodiments disclosed herein, the system operates in a continuous fashion to permit continuous (perfusion) production of the one or more biological molecules or products which allows for the highest cell densities to be achieved as spent media containing toxic metabolite waste is removed and fresh media containing nutrients is added.

In one embodiment, a system is disclosed which includes at least one culture bioreactor, at least one production bioreactor and at least one hydrocyclone (e.g., a hydrocyclone as disclosed herein), wherein the hydrocyclone links the culture bioreactor and the production bioreactor to permit continuous production of the one or more biologic molecules of interest or biologic products, with the hydrocyclone receiving fluid from the culture bioreactor and serving as a cell retention device therefor, then returning the underflow to the culture bioreactor and providing the overflow (a partially cell-depleted fluid) to the production bioreactor.

In one embodiment, the system comprises a single hydrocyclone.

In certain embodiments, the system includes two or more hydrocyclones, for example two or more hydrocyclones operating in parallel with fluid flow generated from a single large pump. In one embodiment, the system includes two or more hydrocyclones, which can be operated in parallel. In certain embodiments, the system includes two or more hydrocyclones of identical geometries. The entire hydrocyclone overflow stream can be used to provide fresh cells to a production bioreactor that is between about 5 to about 20 times by volume larger than the culture reactor, and more particularly, about 5 to about 18 times, about 5 to about 14 times, about 5 to about 10 times, or about 5, about 10, about 15 or about 20 times volume larger than the culture bioreactor.

In certain embodiments, the system may be a stand-alone system (for culturing host cells and in certain embodiments, producing a biologic product in production reactor) or part of a larger system containing one or more additional (e.g., downstream) components. The product of the stand-alone system is one or more biologic molecules of interest in the production bioreaction or optionally, utilizing one or more components to produce a whole cell harvest.

In certain embodiments, the system further comprises one or more downstream components, e.g. a larger system. These downstream components may include, but are not limited to, components for clarifying, capturing, purifying, polishing and/or packaging the product of the production bioreactor(s).

Exemplary products of the system include, but are not limited to a clarified product containing the one or more biologic molecules of interest, a purified product containing the one or more biologic molecules of interest, a polished product containing the one or more biologic molecules of interest, a formulated product containing the one or more biological molecules of interest or a packaged product containing the one or more biologic molecules of interest.

In a particular embodiment, the product of the larger system is a clarified, purified, polished, formulated product containing the one or more biologic molecules of interest.

Generally, during production, the bioreactors described herein comprise host cells (i.e., a plurality of host cells) and medium. The cell culture comprises host cells or cell culture. In certain embodiments, the plurality of host cells are cultured host cells. The host cells can be from any cell source for which culture of the cells is desired, for example, for expansion and/or proliferation of the cells. In one embodiment, the cells are selected from animal, insect, and plant cells. The cell can be genetically engineered, i.e., contains one or more gene sequences whose gene product(s) are produced by the cell.

In one embodiment, the host cell is a mammalian cell. Mammalian cells as used herein are mammalian cells lines suitable for the production of a secreted recombinant therapeutic protein and may hence also be referred to as “host cells”. In certain embodiments, the mammalian cells are rodent cells such as hamster cells. The mammalian cells are isolated cells or cell lines. In certain embodiments, the mammalian cells are transformed and/or immortalized cell lines. In certain embodiments, the mammalian cells are adapted to serial passages in cell culture and do not include primary non-transformed cells or cells that are part of an organ structure. In certain embodiments, the mammalian cells are BHK21, BHK TK⁻, Jurkat cells, 293 cells, HeLa cells, CV-1 cells, 3T3 cells, CHO, CHO-K1, CHO-DXB11 (also referred to as CHO-DUKX or DuxB11), a CHO-S cell and CHO-DG44 cells or the derivatives/progenies of any of such cell line. In certain embodiments, the mammalian cells are CHO cells, such as CHO-DG44, CHO-K1 and BHK21, and even more preferred are CHO-DG44 and CHO-K1 cells. In certain embodiments, the mammalian cells are CHO-DG44 cells. Glutamine synthetase (GS)-deficient derivatives of the mammalian cell, particularly of the CHO-DG44 and CHO-K1 cell are also encompassed. In one embodiment, the mammalian cell is a Chinese hamster ovary (CHO) cell, for example a CHO-DG44 cell, a CHO-K1 cell, a CHO DXB11 cell, a CHO-S cell, a CHO GS deficient cell or a derivative thereof.

In certain embodiments, the mammalian cell may further comprise one or more expression cassette(s) encoding a heterologous protein, such as a therapeutic protein, for example a recombinant secreted therapeutic protein. In certain embodiments, the host cells may also be murine cells such as murine myeloma cells, such as NS0 and Sp2/0 cells or the derivatives/progenies of any of such cell line. Non-limiting examples of mammalian cells which can be used in the meaning of this invention are also summarized in Table 1. However, derivatives/progenies of those cells, other mammalian cells, including but not limited to human, mice, rat, monkey, and rodent cell lines, can also be used in the present invention, particularly for the production of biopharmaceutical proteins.

Examples of mammalian production cell lines are provided in Table A.

TABLE A
Mammalian production cell lines

| Cell line | Order Number |
|---|---|
| NS0 | ECACC No. 85110503 |
| Sp2/0-Ag14 | ATCC CRL-1581 |
| BHK21 | ATCC CCL-10 |
| BHK TK⁻ | ECACC No. 85011423 |
| HaK | ATCC CCL-15 |
| 2254-62.2 (BHK-21 | ATCC CRL-8544 |
| derivative) | |
| CHO | ECACC No. 8505302 |
| CHO wild type | ECACC 00102307 |
| CHO-K1 | ATCC CCL-61 |
| CHO-DUKX | ATCC CRL-9096 |
| (=CHO duk⁻, CHO/dhfr⁻,, | |
| CHO-DXB11) | |
| CHO-DUKX 5A-HS-MYC | ATCC CRL-9010 |
| CHO-DG44 | Urlaub G, et al., 1983. Cell. 33: 405-412. |
| CHO Pro-5 | ATCC CRL-1781 |
| CHO-S | Life Technologies A1136401; CHO-S is |
| | derived from CHO variant Tobey et al. 1962 |
| V79 | ATCC CCC-93 |
| B14AF28-G3 | ATCC CCL-14 |
| HEK 293 | ATCC CRL-1573 |
| COS-7 | ATCC CRL-1651 |
| U266 | ATCC TIB-196 |
| HuNS1 | ATCC CRL-8644 |
| CHL | ECACC No. 87111906 |
| CAP¹ | Wölfel J, et al., 2011. BMC Proc. 5(Suppl |
| | 8): P133. |
| PER.C6 ® | Pau et al., 2001. Vaccines. 19: 2716-2721. |
| H4-II-E | ATCC CRL-1548 |
| | ECACC No.87031301 |
| | Reuber, 1961. J. Natl. Cancer Inst. 26: 891- |
| | 899. |
| | Pitot HC, et al., 1964. Natl. Cancer Inst. |
| | Monogr. 13: 229-245. |
| H4-II-E-C3 | ATCC CRL-1600 |
| H4TG | ATCC CRL-1578 |
| H4-II-E | DSM ACC3129 |
| H4-II-Es | DSM ACC3130 |

¹CAP (CEVEC's Amniocyte Production) cells are an immortalized cell line based on primary human amniocytes.

Examples of mammalian cells, which can be used in the systems and methods described herein include, but are not limited to, cells from the following cell lines: NS0, Sp2/0-Ag14, BHK21, BHK TK⁻, HaK, 2254-62.2 (BHK-21 derivative), CHO, CHO wild type, CHO-DUKX, CHO-DUKX B11, CHO-DG44, CHO Pro-5, CHO-S, Lec13, V79, HEK 293, COS-7, HuNS1, Per.C6, CHO-K1, CHO-K1/SF, CHO-K1 GS, CHOZN GS. In one embodiment, the mammalian cells are selected from the group consisting of: CHO cells, HEK-293 cells, VERO cells, NSO cells, PER.C6 cells, Sp2/0 cells, BHK cells, MDCK cells, MDBK cells, or COS cells. In one embodiment, the mammalian cells are CHO cells.

In certain embodiments, the mammalian cells are being established, adapted, and completely cultivated under serum free conditions, and optionally in media, which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, CA), CHO-S-Invitrogen), serum-free CHO Medium (Sigma), and protein-free CHO Medium (Sigma) are exemplary appropriate nutrient solutions. Any of the media may be supplemented as necessary with a variety of compounds, non-limiting examples of which are recombinant hormones and/or other recombinant growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics and trace elements. Any other necessary supplements may also be included at appropriate concentrations that would be known to those skilled in the art. For the growth and selection of genetically modified cells expressing a selectable gene a suitable selection agent is added to the culture medium.

In a particular embodiment, the cells are cultivated under conditions that allow the cells to proliferate, or under conditions, which are favorable for the expression of the biologic molecule of interest. The biologic molecule of interest is then isolated from the cells and/or the cell culture supernatant. In certain embodiments, the biologic molecule of interest is recovered from the culture medium as a secreted polypeptide, or it can be recovered from host cell lysates if expressed without a secretory signal.

In one embodiment, the host cells are cells which are able to withstand the high pressure drop that occurs when the hydrocyclone is operating under conditions required to achieve reasonable separation efficiencies, such as high-viability cells or cells that are generally more resistant to damage by hydrodynamic shear.

In the disclosed continuous bioreactor system, the cells in the culture bioreactor (e.g., N−1 culture) are typically maintained in a highly proliferative and highly viable state with somewhat high perfusion rates and rates of cell bleed. Generally, the flow rate that any particular hydrocyclone can process is dependent upon its design. Cell bleed rates will also vary and are limited by the cell growth rate. The cell bleed must be less than cell growth rate such that the cell density does not eventually fall to zero (washout). In certain embodiments, steady state perfusion rates are in the range of about 1.05 and 1.4 RV per day and corresponding effective cell bleed rates are in the range of about 0.54 to about 0.70 RV per day. Lower perfusion rates can be used in the start-up (initiation) of the bioreactor prior to reaching a steady state, or when troubleshooting.

In one embodiment, the average viable cell density (VCD) of the culture bioreactor is in the range of about 10×10⁶ to about 40×10⁶, about 10×10⁶ to about 25×10⁶, about 10×10⁶ to about 15×10⁶, about 13×10⁶ to about 18×10⁶, about 15×10⁶ to about 20×10⁶ or about 20×10⁶ to about 25×10⁶ cells/mL.

In one embodiment, the average perfusion rate is in the range of about about 0.1 to about 1.8, about 0.5 to about 1.5, or about 0.8 to about 1.3 RV/day.

Any suitable medium may be utilized in the bioreactor. The media may contain, for example, salts, amino acids, vitamins, lipids, detergents, buffers, growth factors, hormones, cytokines, trace elements and carbohydrates.

Liquid can be added to and/or removed from the bioreactor(s) via any suitable pathway, e.g., a liquid inlet.

The system may be any desired size, for example bench-sized, pilot-sized or manufacturing-sized. In one embodiment, the system disclosed herein is a bench-scale, pilot-scale or manufacturing-scale system. The bioreactors may be any desired size, for example bench-sized, pilot-sized or manufacturing sized.

As defined herein, “working volume” refers to the actual volume of liquid the bioreactor can practically hold while culturing cells. The working volume is less than the total volume of the bioreactor, as there must be room at the top, for example, for bubbles to break up, for vent ports, and the like. In certain embodiments, the working volume is about 50 to about 95%, about 50 to about 90%, about 60 to about 95%, about 60 to about 90%, about 70 to about 95%, or about 70 to about 90% the total volume of the bioreactor. As defined herein, the “working volume” of the system refers to the working volume that the production bioreactor can practically hold curing a culturing process.

The system can have a working volume of about 0.5 to about 25,000 L. In certain embodiments, the system has a working volume of about 0.5 to about 20 L. In certain embodiments, the system has a working volume of about 20 to about 500 L. In certain embodiments, the system has a working volume of about 500 to about 25,000 L. In one embodiment, the system is a bench-scale system having a working volume of about 0.5 to about 20 L. In one embodiment, the system is a pilot-scale system having a working volume of about 20 to about 500 L. In one embodiment, the system is a manufacturing- or production-scale system having a working volume of about 500 to about 25,000 L.

In certain embodiments, the system is a bench-scale system having a working volume of about 0.5 L or more, about 1 L or more, about 5 L or more, about 10 L or more, or about 15 L or more. In certain embodiments, the system is a bench-scale system having a working volume of less than 20 L.

In certain embodiments, the system is a pilot-scale system having a working volume of about 100 L or more, about 200 L or more, about 300 L or more, or about 400 L or more. In certain embodiments, the system is a pilot-scale system having a working volume of less than 500 L.

In certain embodiments, the system is manufacturing-scale system having a working volume of about 500 L or more, about 1000 L or more, about 2000 L or more, about 3000 L or more, about 4000 L or more, about 5000 L or more, about 6000 L or more, about 7000 L or more, about 8000 L or more, about 9000 L or more, about 10,000 L or more, about 15,000 L or more, or about 20,000 L or more. In certain embodiments, the system is a manufacturing-scale system having a working volume of less than 25,000 L.

The production reactor can have a working volume of about 0.5 to about 25,000 L. In certain embodiments, the production reactor has a working volume of about 0.5 to about 20 L. In certain embodiments, the production reactor has a working volume of about 20 to about 500 L. In certain embodiments, the production reactor has a working volume of about 500 to about 25,000 L.

In certain embodiments, the production reactor has a working volume of about 0.5 L or more, about 1 L or more, about 5 L or more, about 10 L or more, or about 15 L or more. In certain embodiments, the production reactor has a working volume of less than 20 L.

In certain embodiments, the production reactor has a working volume of about 100 L or more, about 200 L or more, about 300 L or more, or about 400 L or more. In certain embodiments, the production reactor has a working volume of less than 500 L.

In certain embodiments, the production reactor has a working volume of about 500 L or more, about 1000 L or more, about 2000 L or more, about 3000 L or more, about 4000 L or more, about 5000 L or more, about 6000 L or more, about 7000 L or more, about 8000 L or more, about 9000 L or more, about 10,000 L or more, about 15,000 L or more, or about 20,000 L or more. In certain embodiments, the production reactor has a working volume of less than 25,000 L.

In certain embodiments, the ratio of the working volume of the culture bioreactor (e.g. N−1 bioreactor) to the working volume of the production bioreactor (e.g. N bioreactor) is in the range of about 1:5 to about 1:20, about 1:5 to about 1:10, or about 1:10 to about 1:20. In certain embodiments, the ratio of the working volume of the culture bioreactor (e.g. N−1 bioreactor) to the working volume of the production bioreactor (e.g. N bioreactor) is about 1:5, 1:6, 1:7, 1:8, 1:9, 1:10, 1:11, 1:12, 1:13, 1:14, 1:15, 1:16, 1:17, 1:18, 1:19 or 1:20. For example, if the culture bioreactor has a working volume of about 500 L, and the production bioreactor has a working volume of about 2,500 L, then the ratio of the working volume of the culture bioreactor to the working volume of the production bioreactor is about 1:5.

In certain embodiments, the culture bioreactor has a working volume in the range of about 0.02 L to about 5,000 L, 0.02 L to about 4,000 L, about 0.02 to about 4 L, about 4 to about 100 L, about 100 L to about 4,000 L, or about 100 L to about 5,000 L. In certain embodiments, the culture bioreactor has a working volume of about 0.02 L or more, about 0.04 L or more, about 1 L or more, about 2 L or more, or about 3 L or more. In certain embodiments, the culture bioreactor has a working volume of less than 4 L.

In certain embodiments, the culture bioreactor has a working volume of about 4 L or more, about 20 L or more, about 40 L or more, about 60 L or more, or about 80 L or more. In certain embodiments, the culture bioreactor has a working volume of less than 100 L.

In certain embodiments, the culture bioreactor has a working volume of about 100 L or more, about 200 L or more, about 400 L or more, about 600 L or more, about 800 L or more, about 1000 L or more, about 1200 L or more, about 2,000 L or more, about 3,000 L, or about 4000 L or more. In certain embodiments, the culture bioreactor has a working volume of less than 4,000 L. In certain embodiments, the culture bioreactor has a working volume of less than 5,000 L.

Unlike fed-batch bioreactor operations which generate a single large-volume harvest at the termination of the culture (usually after 10-18 days), the continuous bioreactor system disclosed herein operating at a steady state generates a continuous flow of product from the bioreactor. In certain embodiments, the continuous flow of product from the bioreactor has a consistent titer and product quality.

In a particular embodiment, the production bioreactor exhibits a steady state volumetric productivity of about 0.85 to about 0.96 g/L per day or more.

In one embodiment, the production bioreactor exhibits a steady state volumetric productivity of about 0.96, about 0.97, about 0.98, about 0.99, about 1.00, about 1.01, about 1.02, about 1.03, about 1.04, or about 1.05 g/L per day or more.

In another embodiment, the system exhibits reduced media use relative to a comparable continuous culture system utilizing a membrane cell retention device in place of the one or more hydrocyclones.

In a particular embodiment, the system uses about 0.05 to about 0.8, or about 0.1 to about 0.8 RV/day or less of media or concentrated feed, and more particularly about 0.05, about 0.1, about 0.2, about 0.4, about 0.6 or about 0.8 RV/day (based on the volume of the production reactor).

In a particular embodiment, the system uses about 2 to 3-fold less media and concentrated feed in comparison to the 1.05-1.40 RV/day the N−1 perfusion bioreactor requires as a standalone unit operation.

In a particular embodiment, the system achieves volumetric productivities of about 0.9 g/l/day to about 3 g/l/day, or about 1 g/L/day to about 3 g/L/day. In certain embodiments, the concentration of the antibody leaving the production bioreactor is in the range of about 4 g/L/day to about 6 g/L/day.

Additional benefits of replacing a membrane cell retention device in a comparable conventional system with one or more hydrocyclones as described herein include a significantly reduced risk of fouling or clogging and the ease of operating a small and simple device compared to a large membrane system. In conventional systems with membrane cell retention devices, the membranes may require periodic cleaning. Sanitation and/or sterilization of the system is simplified by replacing a membrane cell retention device in a comparable conventional system with one or more hydrocyclones as described herein. Compared to systems employing membrane cell retention devices, fouling will be minimized or eliminated in the systems described herein which comprise a hydrocyclone.

In one embodiment, compared to membrane cell retention, lower separation efficiencies and shear damage result from multiple passes through a hydrocyclone in the disclosed perfusion system and method. As perfusion rates are increased, more cells are lost to the overflow stream resulting in cellular bleed rates too high for quickly generating the large cell numbers necessary for a production bioreactor inoculation (in the case of N−1 perfusion), or for achieving high cell densities desirable in a continuous perfusion production bioreactor. Also, in order to achieve high volumetric productivities in continuous perfusion production bioreactors, cell-specific productivity should be maximized, which is usually achieved at lower cellular growth rates. In a continuous perfusion bioreactor, conditions that lead to low growth rates with low cell turnover often result in lower cellular viabilities, which may make cells from such a culture unsuitable for perfusion using hydrocyclones due to the shear forces encountered in the device.

In one embodiment, the production bioreactor can operate continuously for duration greater than about 6 weeks. In a particular embodiment, the production bioreactor can operate continuously for greater than about 3 months, greater than about 6 months or greater than about one year. In a particular embodiment, the production bioreactor can operate continuously indefinitely. In certain embodiments, the culture bioreactor can operate continuously for up to about 3 months. In certain embodiments, the culture bioreactor can be restarted as needed (for example, every 3 months) to increase cell productivity.

Culture from the culture bioreactor will enter the production bioreactor at a continuous and fixed flowrate which will be equivalent to the cell bleed rate of the culture bioreactor. Additionally, the production bioreactor will have a continuous feed of nutrient media as the cells in that bioreactor will continue to metabolize, produce product, and potentially undergo some limited cell division. The working volume of both bioreactors would likely be held constant, so the effective dilution rate of the production bioreactor would be determined by the flows of the culture continuous cell bleed and the continuous feed of nutrient media and any pH-controlling titrant added directly to the production reactor. In certain embodiments, the hydrocyclone is continuously operating. In certain embodiments, the hydrocyclone is continuously receiving culture from the culture bioreactor.

In situations where the cell line is not sufficiently genetically stable for long term operation, a second culture bioreactor may be used that would start periodically with an inoculum of fresh cells expanded from frozen vials and once that bioreactor has come up to the proper cell density the second culture bioreactor could take the place of the first culture bioreactor while the first culture bioreactor is taken down for cleaning and re-sterilization. The cell density in the culture bioreactor(s) may vary according to the size of the production bioreactor is it linked to. In some embodiments, the cell density in the culture bioreactor is about 10×10⁶ cells/mL or more, or is about 20×10⁶ cells/mL or more, or is about 40×10⁶ cells/mL or more, or is between 10×10⁶ and 200×10⁶ cells/mL, or is between 40×10⁶ to 120×10⁶ cells/mL or is at a specific density within any of these ranges.

In one embodiment, the system comprises a culture bioreactor (N−1 bioreactor) in which the cells are cultured before they are transferred to a production bioreactor through one or more hydrocyclones. In another embodiment, the system comprises two culture culture bioreactors that operate in alternative to produce cultured cells for transfer to a production bioreactor through one or more hydrocyclones.

In one embodiment, the production bioreactor operates under conditions that promote highest cell productivity. To achieve high productivity conditions, known chemicals that improve per cell productivity but slow or stop growth can be added to the production bioreactor. Alternatively, the bioreactor may be made to operate, for example, under low or high pH, or low temperature (with additions of Cu, low Ca, additions of galactose, etc.) that are beneficial for getting cells to produce protein with particular quality attributes, but those same conditions do not allow for high rates of cell growth.

A production bioreactor operating at a high dilution rate may result in higher growth rates as inhibitory metabolites would be more effectively flushed out of the bioreactor. The dilution rate of the production bioreactor will be dependent upon the cell bleed rate from the N−1 bioreactor, and the rate of feed and any titrant used for pH control directly entering the production bioreactor. Manipulating the concentration of the feed medium to the production bioreactor would allow for a balance of optimal growth, nutrient availability, and inhibitor metabolite flushing from the system. In certain embodiments, the dilution rate of the production bioreactor is adjusted by using a single concentrated feed media, and to dilute this media with water, or water mixed with a saturated saline solution as needed. Control of the dilution rate with variable concentration feed media might also allow for control over the residence time of the recombinant protein produced from the system. High dilution rates would result in low product residence times and might be advantageous for highly labile proteins.

The subject technology relates to a biologic molecule of interest, or a protein of interest, produced by a method as described above. Such a biologic molecule or protein of interest includes, but is not limited to a biopolymer; an antibody, such as monoclonal antibody; a fusion protein, such as a Fc-fusion proteins; enzymes, cytokines, such as interleukins (IL); lymphokines; adhesion molecules; insulin; insulin-like growth factor; hGH; tPA; virus; virus-like particle; adeno-associated virus; receptors and derivatives or fragments thereof; and any other polypeptides and scaffolds that can serve as agonists or antagonists and/or have therapeutic or diagnostic use.

In one embodiment, the biologic molecule of interest is an antibody or a fragment or derivative thereof. The systems and methods described herein can be advantageously used for production of antibodies such as monoclonal antibodies, multispecific antibodies, or fragments thereof, preferably of monoclonal antibodies, bi-specific antibodies or fragments thereof. Antibody fragments include e.g. “Fab fragments” (Fragment antigen-binding=Fab). Fab fragments consist of the variable regions of both chains, which are held together by the adjacent constant region. These may be formed by protease digestion, e.g. with papain, from conventional antibodies, but similar Fab fragments may also be produced by genetic engineering. Further antibody fragments include F(ab′)2 fragments, which may be prepared by proteolytic cleavage with pepsin.

Using genetic engineering methods, it is possible to produce shortened antibody fragments which consist only of the variable regions of the heavy (VH) and of the light chain (VL). These are referred to as Fv fragments (Fragment variable=fragment of the variable part). Since these Fv-fragments lack the covalent bonding of the two chains by the cysteines of the constant chains, the Fv fragments are often stabilized. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins are known to the person skilled in the art. Preferred secreted recombinant therapeutic antibodies according to the invention are bispecific antibodies. Bispecific antibodies typically combine antigen-binding specificities for target cells (e.g., malignant B cells) and effector cells (e.g., T cells, NK cells or macrophages) in one molecule. Exemplary bispecific antibodies, without being limited thereto are diabodies, BiTE (Bi-specific T-cell Engager) formats and DART (Dual-Affinity Re-Targeting) formats. Also anticipated in the context of the present invention are minibodies. By minibody, the skilled person means a bivalent, homodimeric scFv derivative.

In certain embodiments, the biologic molecule of interest is recovered or isolated from the culture medium as a secreted polypeptide. In order to isolate the biologic molecule of interest from other recombinant proteins and host cell proteins to obtain substantially homogenous preparations of the biologic molecule of interest, cells and/or particulate cell debris are removed from the culture medium or lysate. Next, the biologic molecule of interest is purified from contaminant soluble proteins, polypeptides and nucleic acids, for example, by fractionation on immunoaffinity or ion-exchange columns, ethanol precipitation, reverse phase HPLC, Sephadex chromatography, and chromatography on silica or on a cation exchange resin such as DEAE. Methods for purifying a heterologous protein expressed by host cells are known in the art.

In one embodiment, the system further comprises one or more downstream components. In a particular embodiment, the one or more downstream components are selected from precipitation components, purification components, finishing components, packaging components or combinations thereof. In one embodiment, the precipitation and/or the purification components are chromatography columns.

In certain embodiments, at least one downstream component is not linked to the system, i.e., a hybrid system. In one embodiment, one or more of the downstream elements are not linked to the other elements of the continuous system, i.e., a hybrid system.

In certain embodiments, all of the downstream components are linked to the system, i.e., a continuous end-to-end system.

In one embodiment, the one or more downstream elements are linked to the other elements of the continuous system, i.e., a continuous end-to-end system.

In a particular embodiment, the system comprises of a highly proliferative culture bioreactor which supplies cells to a production bioreactor having a working volume of about 2,000 to about 10,000 L or more. In a particular embodiment, the system would operate at a steady state volumetric productivity as high as 0.96 g/L/day, require only 0.17 RV/day of media, and deliver a continuous stream of antibody product at 5.7 grams/L.

### Culture Bioreactor

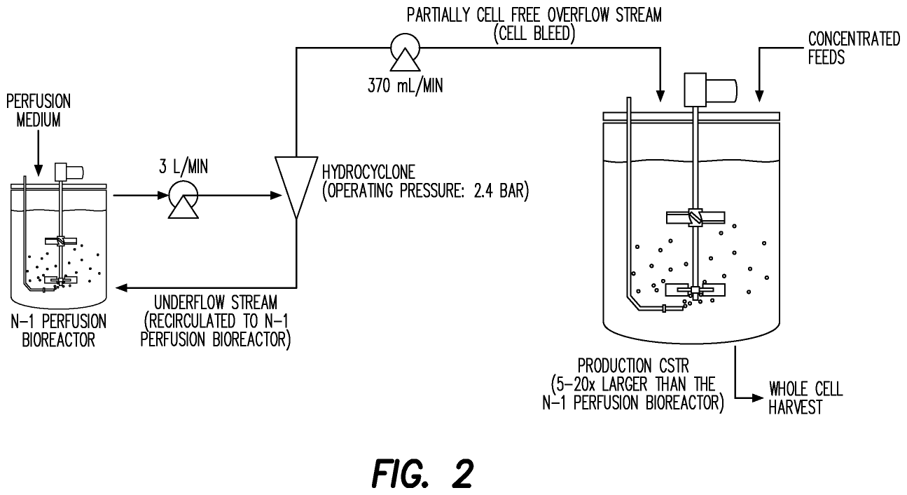

In certain embodiments, the system disclosed herein includes at least one culture bioreactor for culturing the host cells (e.g., mammalian host cells) that will be used for inoculating the production bioreactor. The culture bioreactor is linked (i.e., in fluid communication with) a hydrocyclone, for example, as shown in FIG. 2.

In one embodiment, the culture bioreactor is a N−1 bioreactor or a N−1 perfusion bioreactor.

The volume of the culture bioreactor may vary. In one embodiment, the volume of the culture bioreactor is significantly less than the production bioreactor. In a particular embodiment, the volume of the culture bioreactor is 500 L.

As a unit, the culture bioreactor may be continuous. In one embodiment, the culture bioreactor may be a perfusion culture bioreactor or perfusion bioreactor, may be continuous or semi-continuous with respect to liquid transfer to and/or from the culture bioreactor. In a particular embodiment, the cell bleed or transfer from the culture bioreactor to the production bioreactor may be continuous or semi-continuous, indirectly by means of overflow from the hydrocyclone.

In certain embodiments, the culture (e.g., perfusion) bioreactor is configured to produce and maintain a host cell culture. In certain embodiments, the culture (e.g., perfusion) bioreactor comprises a reaction chamber and one or more ports or connections to permit entry or exit of liquid.

In one embodiment, the culture (e.g. perfusion) bioreactor contains a plurality of host cells (e.g., cultured host cells such as cultured mammalian host cells) and cell medium.

The viability of the host cells in the culture bioreactor may vary. In a particular embodiment, the cell viability is about 50 to about 100%, about 60 to about 100%, about 70 to 100%, about 80 to 100%, about 90 to 100%, about 60%, about 65%, about 70%, about 75%, about 80%, about 85%, about 90%, about 95% or about 100%.

In a particular embodiment, the viable cell density is between about 20×10⁶ and about 30×10⁶ cells/mL.

The culture reactor conditions will depend on the cell type (e.g., dissolved gas concentrations, temperature, pH) and will be adjusted for optimal growth and productivity of the cell as necessary, which will be apparent to those skilled in the art. In certain embodiments, the pH is in the range of about 6.5. to about 7.5. In certain embodiments, the temperature is in the range of about 27 to about 38° C., or about 31 to about 37° C. In certain embodiments, the dissolved oxygen is in the range of about 10 to about 80% of air saturation.

After culturing, a quality of the cultured host cells is transferred (bled) to the production bioreactor, which is optionally linked (i.e., in fluid communication with) the culture bioreactor. In certain embodiments, the cultured host cells are transferred (bled) to the production bioreactor as a partially cell-depleted fluid in the form of overflow from a hydrocyclone which links the culture bioreactor to the production bioreactor and serves as a cell retention device.

In one embodiment, envisioned cell transfer (bleed) to the production CSTRs begins as soon as perfusion is started.

In a particular embodiment, the rate of cell bleed/transfer from the culture bioreactor to the production bioreactor is from about 0.1 reactor volumes per day (RV/day) to about 1.3 RV/day, about 0.1 to about 1.4 RV/day, about 0.1 to about 1.5 RV/day, or about 0.1 to about 2.0 RV/day. In another embodiment, the rate of cell bleed/transfer from the culture bioreactor to the production bioreactor, either in continuous or semi-continuous mode, is less than 2.0 RV/day, less than 1.5 RV/day, less than 1.4 RV/day, less than 1.3 RV/day, less than 1.0 RV/day, less than 0.8 RV/day, less than 0.6 RV/day, less than 0.4 RV/day, less than 0.2 RV/day.

In one embodiment, the inoculation density is about 1.5×10⁶ cells/mL. In one embodiment, the inoculation density is in the range of about 1×10⁶ to about 2×10⁶ cells/mL.

A. Production Bioreactor

The system disclosed herein also includes a production bioreactor for receiving the inoculum of cultured host cells from the culture bioreactor, culturing the transferred host cells and producing the at least one biologic molecule of interest.

In one embodiment, the production bioreactor is indirectly linked (i.e., in fluid communication) with the culture bioreactor which provides the production bioreactor with an inoculum of cultured host cells in the form of overflow from a hydrocyclone which directly links the culture reactor and the production reactor as an intermediate component of the system, serving as a cell retention device for the culture bioreactor, such that the overflow is a partially cell-depleted fluid.

The production bioreactor is configured to promote the growth and maintenance of at least a first type of host cell suitable (either inherently or by genetic manipulation) to express at least one biologic molecule of interest. In certain embodiments, for example, the production bioreactor comprises a reaction chamber, configured to maintain a suspension comprising a cell culture medium (e.g., a growth cell culture medium configured to promote growth of the first type of host cell, a production cell culture medium configured to promote expression of the at least one biologic molecule of interest) and the first type of host cells. In certain embodiments, the production bioreactor is configured to promote growth and maintenance of one type of host cell suitable to express at least one biologic molecule of interest.

In certain embodiments, the production bioreactor is configured to promote growth and maintenance of two or more types of host cells suitable to express two or more types of biologic molecules of interest. In embodiments wherein the production bioreactor is configured to promote growth and maintenance of two or more types of host cells, the system comprises two or more culture reactors, wherein each culture reactor is configured to culture one type of host cell (i.e., each culture reactor cultures a different type of host cell) and all culture reactors feed into the same production reactor.

In one embodiment, the production bioreactor contains a plurality of host cells (e.g., cultured host cells such as cultured mammalian host cells) and cell medium.

The density of the host cells in the production bioreactor may vary. In certain embodiments, the density of host cells is in the range of about 5×10⁶ to about 200×10⁶ cells/mL, or about 30×10⁶ to about 80×10⁶ cells/mL.

The production reactor conditions will depend on the cell type (e.g., dissolved gas compositions, temperature, pH) and will be adjusted for optimal growth and productivity of the cell as necessary, which will be apparent to those skilled in the art. In certain embodiments, the pH is in the range of about 6.5. to about 7.5. In certain embodiments, the temperature is in the range of about 27 to about 38° C., or about 31 to about 37° C. In certain embodiments, the dissolved oxygen is in the range of about 10 to about 80% of air saturation.

In one embodiment, the production bioreactor operates continuously for a period of greater than 3 weeks; greater than 4 weeks; greater than 5 weeks; or greater than 6 weeks.

In one embodiment, the production bioreaction exhibits increased steady state volume productivity relative to a comparable system utilizing a conventional membrane host cell retention device.

In certain embodiments, the production bioreactor exhibits a steady state volumetric productivity of about 0.5 to about 6 g/L per day, about 1 to about 6 g/L per day, or about 0.5 to about 3 g/L per day. In a particular embodiment, the production bioreactor exhibits a steady state volumetric productivity of about 0.5, about 0.6 or about 1 g/L per day or more.

In one embodiment, the production bioreactor has a volumetric productivity of at least 0.6 grams per liter per day for a period of at least 14 days; the production bioreactor has a volumetric productivity of at least 0.6 grams per liter per day for a period of at least 20 days; the production bioreactor has a volumetric productivity of at least 0.6 grams per liter per day for a period of at least 30 days; the production bioreactor has a product residence time of about 1 to about 10 days; the production bioreactor has a dilution rate of about 1 to about 0.1 volume per day; the production bioreactor is fed with a diluent solution; the diluent solution is water or saline.

In certain embodiments, the production bioreactor has a product residence time of about 1 to about 10 days; the production bioreactor has a dilution rate of about 1 to about 0.1 volume per day. In certain embodiments, no additional diluent (such as water or saline) is used.

In certain embodiments, the production bioreactor has a product residence time of about 1 to about 10 days; the production bioreactor has a dilution rate of about 1 to about 0.1 volume per day; the production bioreactor is fed with a diluent solution; the diluent solution is water or saline.

In certain embodiments, the production bioreactor comprises an outlet (e.g., a peristaltic pump) which permits the removal of which removed whole cell harvest from the bioreactor in response to an increase in bioreactor weight.

### Additional System Components

In one embodiment, the system comprises one or more additional components.

In a particular embodiment, the bioreactor system comprising the culture bioreactor, the production bioreactor and the hydrocyclone is part of a larger system that includes one or more downstream components.

The downstream components may be, for example, components for clarifying, capturing, purifying, polishing and/or packaging the biologic molecule of interest, produced in the production reactor. There may be two or more components of each type, e.g., two or more purifying components, for example, two or more purifying chromatography columns.

In one embodiment, the system is part of a larger system that includes one or more downstream components, wherein at least one of the downstream components is not linked to the system, i.e., a hybrid system.

In another particular embodiment, the system is part of a larger system that includes one or more downstream components, wherein all of the at least one downstream components are linked to the system, i.e., a continuous end-to-end system.

Optionally, one or more components can be removed from the at least one culture reactor and/or the at least one production bioreactor during operation thereof, e.g., impurities, medium or host cells.

### Methods

Disclosed herein are methods of culturing host cells using the systems and methods disclosed herein (or the hydrocyclone in any suitable system) and methods of producing at least one biologic molecule of interest from such cultured host cells.

In one embodiment, a method for producing a biologic molecule of interest is disclosed, the method comprising: (i) culturing a plurality of host cells capable of producing a biologic molecule of interest (e.g., comprising a gene that encodes a protein of interest, e.g. a recombinant protein) in at least one culture bioreactor (for example, an N−1 bioreactor or N−1 perfusion bioreactor (ii) inoculating at least one production bioreactor (for example, an N bioreactor or a continuously stirred tank reactor (CSTR) production bioreactor) with cells obtained from step (i); and (iii) culturing the cells in the production bioreactor, wherein the culture bioreactor and the production bioreactor are linked by at least one hydrocyclone that serves as a cell retention device for the at least one culture bioreactor.

In another embodiment, a method for producing a biologic molecule of interest is disclosed, the method comprising: (i) culturing a plurality of host cells (e.g., mammalian cells) in at least one culture bioreactor (for example, an N−1 bioreactor or N−1 perfusion bioreactor) (ii) inoculating at least one production bioreactor (for example, an N bioreactor or a continuously stirred tank reactor (CSTR) production bioreactor) with cells obtained from step (i); and (iii) culturing the cells in the production bioreactor under conditions that allow production of a biologic molecule of interest; wherein the culture bioreactor and the production bioreactor are linked by at least one hydrocyclone that serves as a cell retention device for at least one culture bioreactor, wherein the hydrocyclone produces an underflow stream containing concentrated cell culture and a partially cell-free overflow stream, and wherein underflow stream containing concentrated cell culture is returned to the culture bioreactor and partially cell-free overflow stream is directed to the production bioreactor. The overflow functions as an inoculum for the production reactor, in the form of a quantity of host cells to be cultured therein.

An exemplary method is shown in FIG. 2.

In one embodiment, the inoculation in step (ii) is by transferring cells from the culture bioreactor to the production bioreactor. In a particular embodiment, the cell transfer is by cell bleed in continuous or semi-continuous modes. In one embodiment, the cell transfer is in semi-continuous mode involving cell transfer once at every period of time from 2 minutes to 24 hours or at any interval therebetween.

Generally, the systems for use in the methods do not comprise a conventional cell retention device such as a membrane cell retention device. In one or more embodiments, the one or more hydrocyclones are used in the method in place of a conventional cell retention device, for example a screen or membrane separation device. In certain embodiments, the one or more hydrocyclones receives a cell culture stream from the culture bioreactor, for example, through inlets at the top of the one or more hydrocyclones, while underflow stream containing concentrated cell culture is returned to the culture bioreactor and partially cell-free overflow stream is directed to the production bioreactor.

In a particular embodiment, the one or more hydrocyclones receive a host cell transfer from the culture bioreactor once at every period of time from 2 minutes to 24 hours or at any interval therebetween. In a particular embodiment, the production reactor receives a host cell transfer from the culture bioreactor by way of the hydrocyclone overflow once at every period of time from 2 minutes to 24 hours or at any interval therebetween.

In one embodiment, the method further comprises one or more additional steps. In a particular embodiment, the method further comprising step (iv) harvesting the protein of interest from the production bioreactor. Other additional steps include downstream steps including, for example, clarification steps, capture steps, purification steps, polishing steps, formulation steps, packaging steps or a combination thereof. These one or more additional steps may be linked to the cell culture method to provide a continuous end-to-end method or one or more of the additional steps may be unlinked to provide a hybrid method.

In a one embodiment, the volume ratio of the culture bioreactor to the production bioreactor is about 1:1 to about 1:20; volume ratio of the culture bioreactor to the production bioreactor is about 1:1 to about 1:5; volume ratio of the culture bioreactor to the production bioreactor is about 1:5.

The following examples are presented for illustrative purposes only and are not intended to be limiting.

### EXAMPLES

### Example 1—Use of Hydrocyclone as Cell Retention Device for N−1 Perfusion Bioreactor Linked to Continuous-Flow Stirred-Tank Bioreactor (CSTR) Operating as Production Bioreactor

FIG. 2 shows the diagram of the two-stage linked-bioreactor system used in this experiment. The N−1 perfusion bioreactor was a 15-L glass vessel (Applikon, Schiedam, Netherlands) operating at 8-L working volume. The cell retention device for perfusion was a hydrocyclone that was 3D printed using an inverted stereolithography printer (Form 2, Formlabs, Somerville, MA). Hydrocyclones with varying underflow diameters (between 2 and 4 millimeters (mm)) and overflow diameters (between 1 and 3 mm) were tested in short duration, non-sterile benchtop experiments.

The geometry in Table 1 and FIG. 3 was chosen because it provided the greatest separation efficiency with minimal impact to cell viability in these tests. The hydrocyclone was 3D printed using an autoclavable and biocompatible resin (Dental SG Vi, Formlabs, Somerville, MA) and cured with ultraviolet light (FormCure, Formlabs, Somerville, MA).

TABLE 1
Hydrocyclone dimensions

| Parameter | Notation | Length (mm) |
|---|---|---|
| Chamber diameter | D_{c} | 10 |
| Cylindrical section length | L_{c} | 4 |
| Conical section length | Lₛ | 71 |
| Vortex finder length | Lᵥ | 2 |
| Vortex finder inner diameter | Dᵥᵢ | 2 |
| Vortex finder wall thickness | W | 0.5 |
| Underflow diameter | Dᵤ | 3 |
| Overflow diameter | Dₒ | 2 |
| Underflow outlet length | Lᵤ | 30 |
| Overflow outlet length | Lₒ | 36 |
| Inlet initial diameter | Dᵢᵢ | 8.5 |
| Inlet final diameter | D_{if} | 2 |
| Inlet length | Lᵢ | 30 |

A peristaltic pump (600 series, Watson Marlow, Wilmington, MA) supplied flow from the N−1 bioreactor through 9.5-mm inner diameter (ID) tubing to the hydrocyclone at approximately 3 liters per minute (L/min) to create a pressure drop of 2.4-2.6 bar within the device. A stream of concentrated cells returned to the N−1 bioreactor in an unrestricted underflow stream. The underflow return loop consisted of approximately 15 centimeters (cm) of 25.4-mm ID tubing which fit over the lower portion of the hydrocyclone. This 25.4 mm ID tubing was reduced to 9.5 mm ID tubing over 4 cm using a 3D printed dual hose barb reducer which was 3D printed using the materials and methods described previously. The 9.5 mm ID tubing was attached to the reducer and welded onto the bioreactor. A second peristaltic pump (500 series, Watson Marlow, Wilmington, MA) controlled the partially cell-free overflow stream leaving the N−1 bioreactor/hydrocyclone system through 9.5-mm ID tubing at a flow rate of 370 milliliters per minute (mL/min) for a maximum daily perfusion volume of 532 L.

Although the overflow stream was restricted with the peristaltic pump for purposes of accurate control, earlier experiments showed the flowrate of an unrestricted overflow stream would have been nearly identical.

In order to use only a fraction of the maximum perfusion capacity, the perfusion rate was controlled by limiting the amount of time the hydrocyclone was operated. For the 8-L N−1 perfusion in this experiment, the hydrocyclone was operated for 30 minutes per day or less. Electrical power outlet timers were used to turn the inlet and overflow pumps on for a few minutes and then off again once every 3 hours. The daily hydrocyclone operational time was thus distributed evenly among the 8 times. Once the pumps turned on, the hydrocyclone system reached hydrodynamic equilibrium within a few seconds and this startup time was considered negligible for the purposes of this experiment. To reduce the amount of cell culture fluid with cells remaining in the recirculation loop in the 3 hours between power cycles, fresh perfusion medium was added semi-continuously (delivered in equivalent doses every fifteen minutes) through connections just prior to the hydrocyclone inlet pump and immediately after the hydrocyclone in the underflow stream. This setup allowed most of the culture in the recirculation loop to be washed back into the bioreactor with fresh medium between power cycles.

The production CSTRs initially only contained basal medium at 70% of the final working volume. Upon startup of the CSTRs a fraction of the overflow stream gradually supplied cells to each CSTR using peristaltic pumps (300 series, Watson Marlow, Wilmington, MA). These supply pumps followed a similar intermittent operating schedule as the inlet and overflow pumps. In order to ensure that the cells being supplied to the production CSTRs were fresh, the pumps supplying cells to the CSTRs were timed to start one minute after the hydrocyclone started operating. During this one-minute startup time, the entire overflow stream was directed to waste. Once the working volume of each CSTR had accumulated to 1.4 liters, the bioreactor working volume was maintained constant by a ChemTec pump (Parker Domnik Hunter, Oxnard, CA) which removed whole cell harvest from the bioreactor in response to an increase in bioreactor weight.

Table 2 shows some details of the media and feeds used in this experiment and Table 3 shows various bioreactor operating parameters.

TABLE 2
The compositions of the media and feed in the
N-1 perfusion and production CSTR bioreactors

| | Total | | | |
|---|---|---|---|---|
| | Amino Acid | Glucose | Osmolality | |
| Medium | (mM) | (g/L) | (mOsm/kg) | pH |
| N-1 and CSTR basal medium | 120 | 4 | 280 | 7.1 |
| N-1 perfusion/dilution | 90 | 5 | 320 | 7.2 |
| medium | | | | |
| CSTR concentrated feed | 600 | 50 | 1080 | 7.2 |
| medium | | | | |

TABLE 3
Operating parameters for the N-1 perfusion and production CSTR bioreactors

| Parameter | N-1 perfusion | Production CSTR Bioreactors |
|---|---|---|
| Inoculation | 1.5 × 10⁶ | 0 |
| Density (viable | | |
| cells/mL) | | |
| Temperature (° C.) | 36.5 | 36.5 |
| pH setpoint | 7.08 ± 0.05 (controlled by 1M | 7.08 + 0.05 (controlled by 1M |
| during HIPDOG | sodium/potassium carbonate on the | sodium/potassium carbonate on |
| | low end and 500 g/L glucose on the | the low end and 500 g/L glucose |
| | high end) | on the high end) |
| pH setpoint post | N/A | 7.05 ± 0.10 (controlled by 1M |
| HIPDOG | | sodium/potassium carbonate on |
| | | the low end and CO₂ on the high |
| | | end) |
| Dissolved oxygen | 40 | 40 |
| setpoint (% air | | |
| saturation) | | |
| Dissolved CO2 | 4-10 | 4-10 |
| (% of saturation) | | |
| Working volume | 8 | 1.4 |
| (L) | | |
| Agitation | Lower 7.6 cm diameter Rushton | Single 4.5 cm diameter Rushton |
| | and upper down-pumping 7.6 cm | impeller at 80 W/m³ power/unit |
| | diameter pitched-blade impellers at | volume |
| | 80 W/m³ power/unit volume | |

The N−1 and production bioreactors utilized a dual sparging strategy to control dissolved oxygen and dissolved carbon dioxide (CO2). The 15-L N−1 perfusion bioreactor was equipped with a 100 m sintered steel sparger which supplied pure oxygen to the culture to control dissolved oxygen. The 15-L N−1 perfusion bioreactor also sparged air through a 7×1 mm drilled hole sparger primarily for CO2 removal. The 3-L production CSTRs initially delivered oxygen to the culture with the 7×1 mm drilled hole sparger. Once the oxygen demand increased to high levels in the production CSTRs, a 15 m sintered steel sparger was used to supply pure oxygen to the culture while pure oxygen continued to sparge through the 7×1 mm drilled hole sparger for both CO2 removal and oxygen delivery. Gases and pH were measured offline using a blood gas analyzer (Siemens Diagnostics, Deerfield, IL). The gas flow rate through the drilled hole spargers were occasionally manually adjusted to maintain the dissolved CO2 within the ranges listed in Table 3.

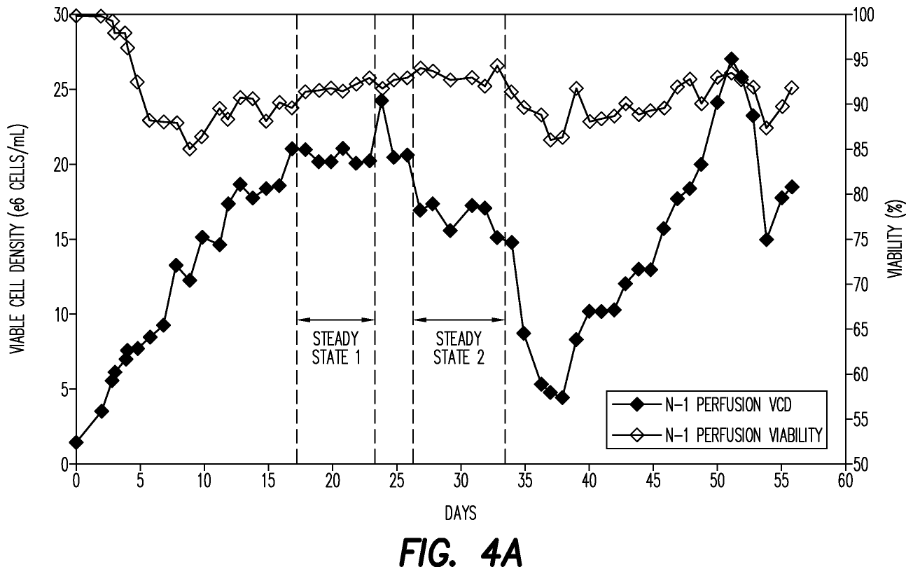

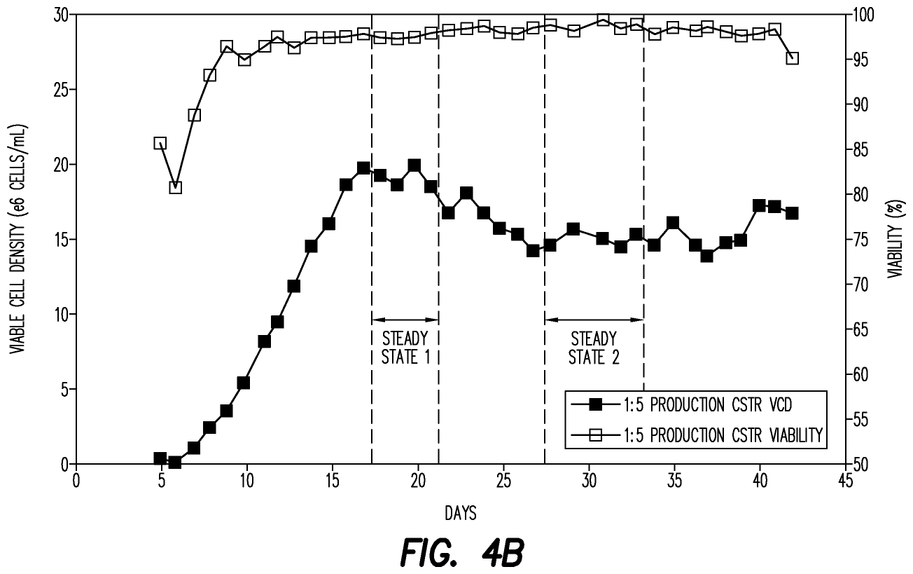

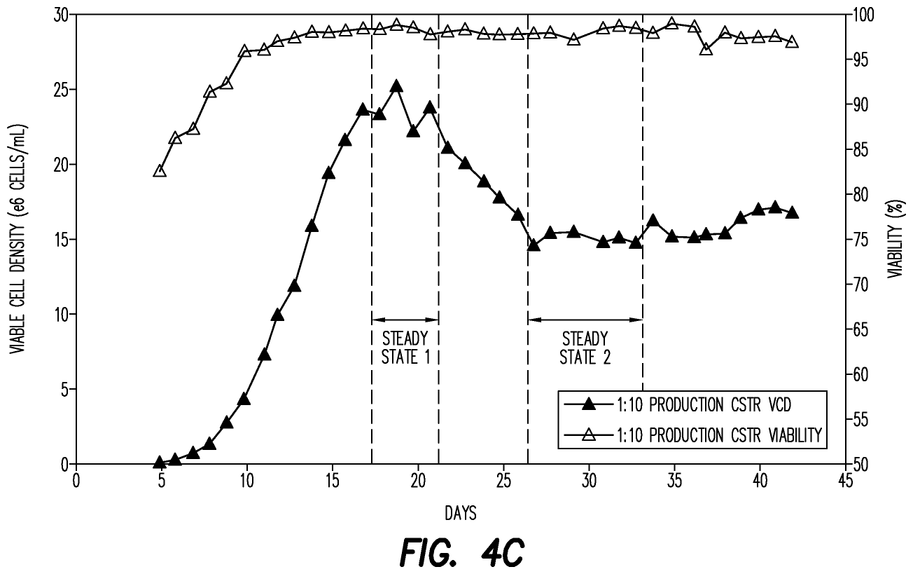

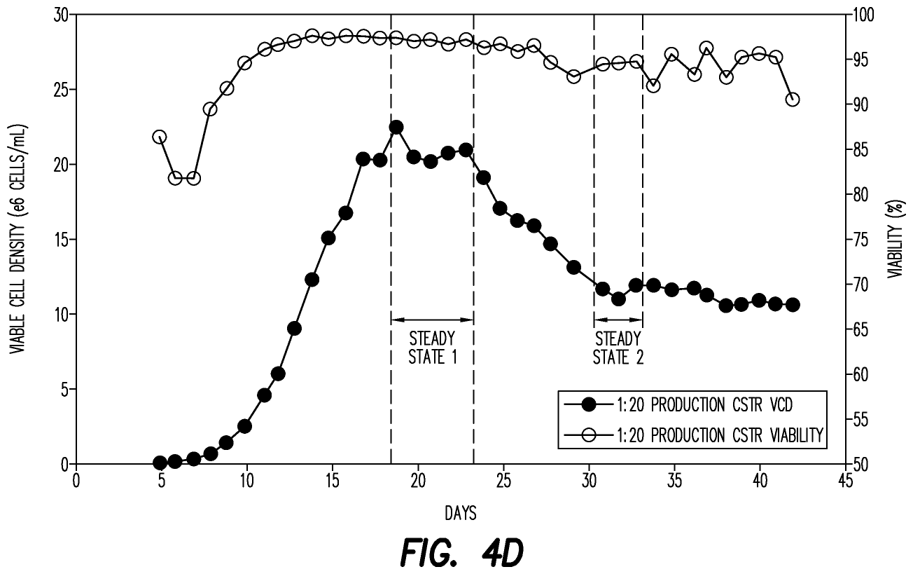

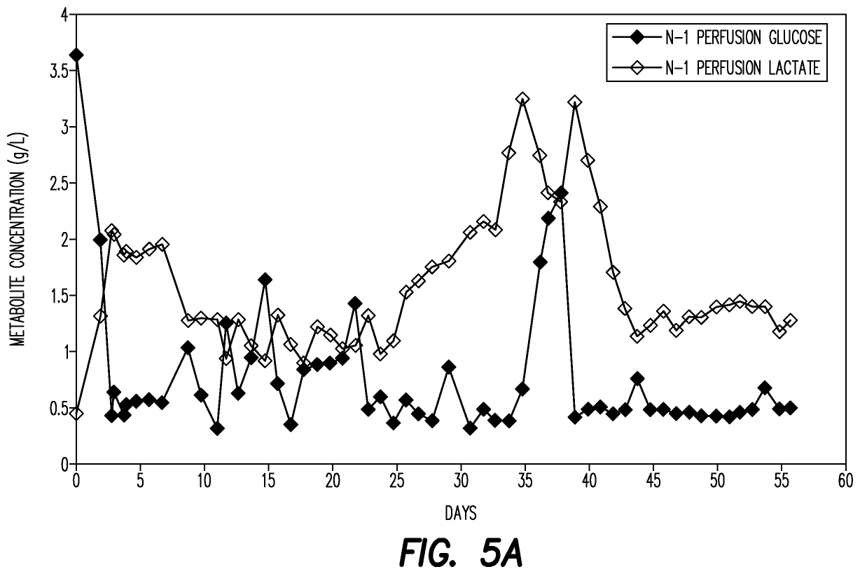

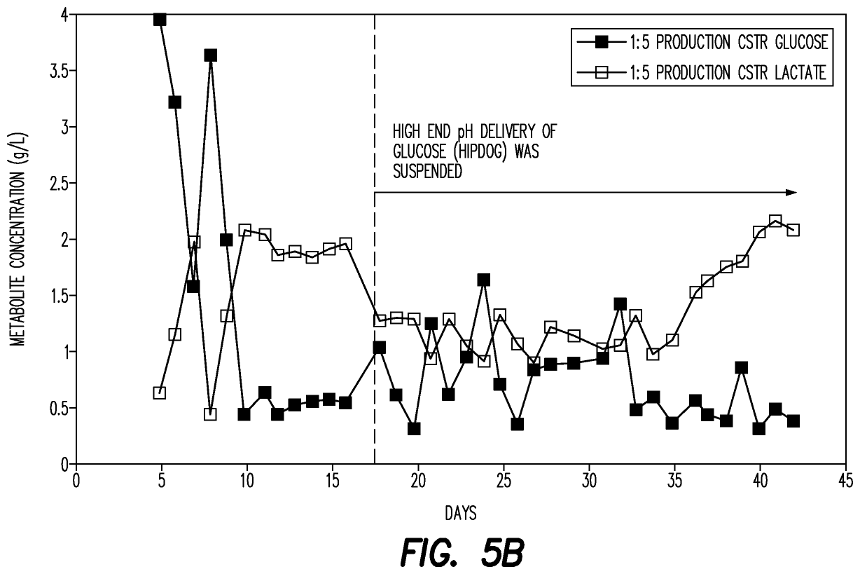

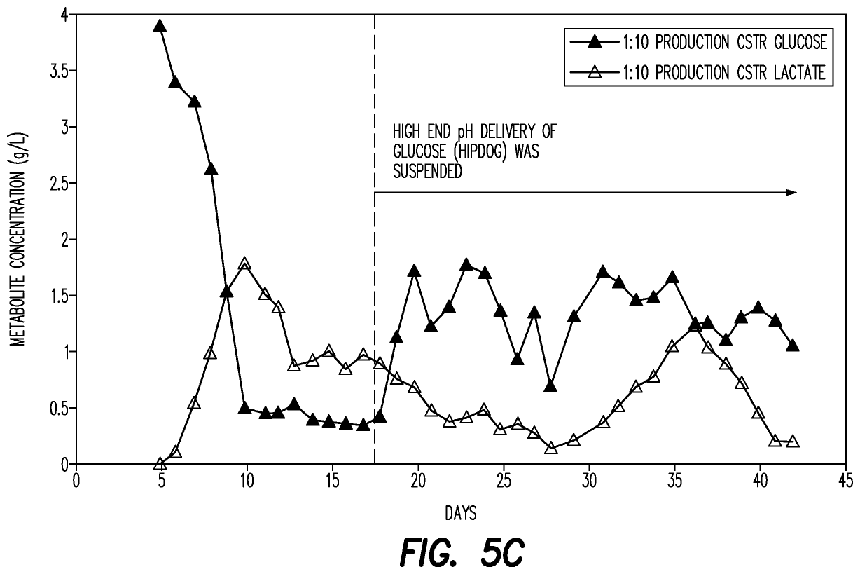

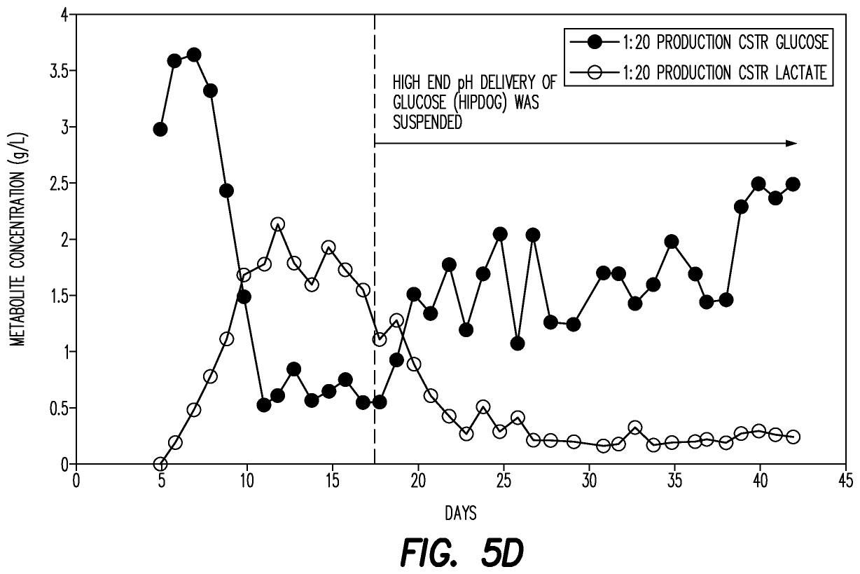

The N−1 perfusion bioreactor was inoculated at 1.5×10⁶ cells/mL and the viable cell density and cell viability trends are shown in FIG. 4a. Viable cell density, cell viability, and metabolites were measured using a NovaFLEX Analyzer (Nova Biomedical, Waltham, MA). In order to control lactate accumulation in the bioreactor, a glucose delivery technique known as High-End pH Delivery of Glucose (HIPDOG) was employed to feed glucose based on pH starting on Day 2 and continued for the duration of the experiment. In this control scheme, the cell culture first depleted the glucose in the basal medium and produced lactic acid. When the cells were glucose limited, they began to consume lactic acid which resulted in a rise in pH. Once the pH increased above the high-end pH setpoint, a glucose pump was triggered and added 500 g/L glucose to the bioreactor. As the culture converted the glucose to lactic acid, the pH was pushed below the high-end setpoint and triggered the glucose pump to turn off. In a perfusion system, lactate is flushed from the bioreactor as a result of the perfusion process itself. Consequently, lactate exhaustion could presumably cause HIPDOG control to fail because without some residual lactate, there would not be a pH response to signal glucose depletion. To prevent HIPDOG failure, a 1M sodium/potassium carbonate solution (molar ratio 0.94M sodium: 0.06M potassium) was added to the bioreactor to generate an upward driving force on pH to ensure that lactate would not be fully depleted. The 1M sodium/potassium carbonate solution was added semi-continuously (as described earlier) at a rate of less than 1% of the perfusion volume and it was adjusted to maintain the residual lactate concentration in the bioreactor between 0.5 and 2 g/L. It should be noted that at perfusion rates higher than 1.2 RV/day, the perfusion medium supplied enough glucose to the culture without additional glucose supplementation by HIPDOG control. Residual glucose and lactate profiles for the N−1 perfusion are shown in FIG. 5a. During steady state timepoints, ammonium was maintained below 6 mM and osmolality was maintained between 280 and 315 mOsm/kg.

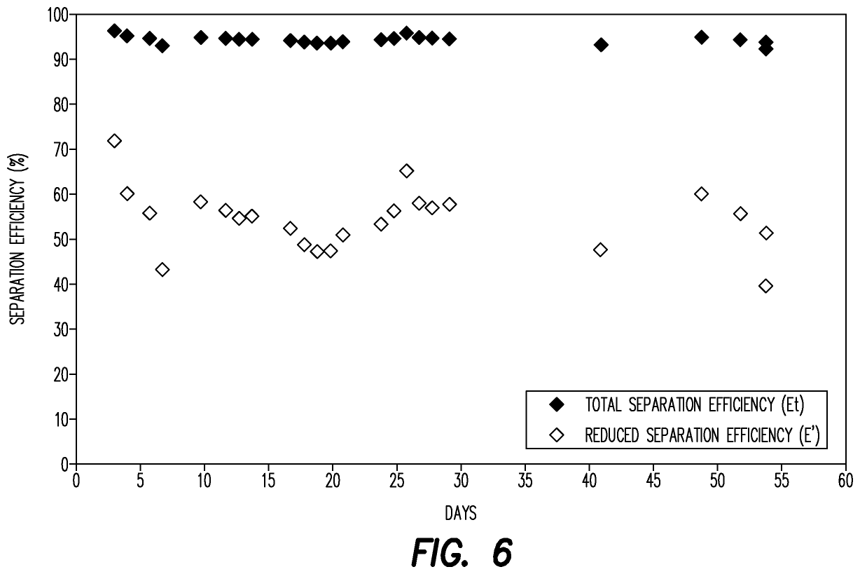

Perfusion was initiated on Day 3 at a cell specific perfusion rate (CSPR) of 50 picoliters per cell per day (pL/cell/day) on Days 3-24. The perfusion rate was adjusted daily to reflect changes in cell density. In previous perfusion experiments, it was determined that this CSPR flushes growth inhibitors (other than lactate) at a sufficient rate to maintain exponential growth for this cell line. The hydrocyclone operating pressure was initially set to 2.6 bar because this condition yielded the highest separation efficiency with a negligible drop in cell viability in short duration tests. However, during the first three days of perfusion, the growth rate dropped significantly, and the cell viability began to trend downward, reaching as low as 85%. On Day 6, the operating pressure was decreased to 2.4 bar in an attempt to reduce shear damage and recover the culture. Although the separation efficiency decreased slightly, the culture quickly returned to near exponential growth and the viability stabilized at 88-94%. Operating at 2.4 bar, 92-96% of the cells that entered the hydrocyclone were recovered in the underflow stream returning to the bioreactor as shown in FIG. 6. This value was determined by calculating the total separation efficiency (Eₜ) using the following equation:

$${E}_{t} = ( 1 - \frac{{X}_{O} {Q}_{O}}{{X}_{brx} Q} ) * 100 \%$$

where X_{O} and X_{brx} denote the viable cell density of the overflow stream and bioreactor, respectively, and Q_{O} and Q denote the flow rates of the overflow stream and inlet stream, respectively. Reduced or centrifugal separation efficiency (E′) can also be calculated using the equation below:

$${E}^{′} = ( 1 - \frac{{X}_{O}}{{X}_{brx}} ) * 100 \%$$

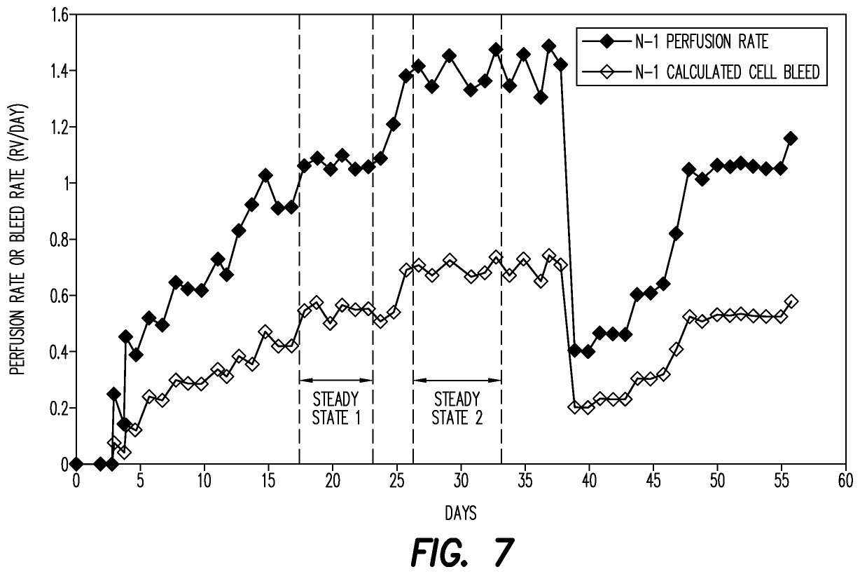

In this experiment, an average reduced separation efficiency of 54% was observed. Since the hydrocyclone does not provide a perfect separation, as the perfusion rate increased, the effective cell bleed from the overflow stream also increased as shown in FIG. 7. As defined herein, the effective bleed rate is the rate of cell bleed that would occur if the cell bleed was being removed directly from the bioreactor on a continuous basis. This cell bleed rate is then equivalent to that typically defined in perfusion bioreactor operation when the cell retention system is 100% effective (as a membrane cell retention system would be) and it is necessary to remove cells directly from the culture in order to achieve a steady-state (biomass constant) condition. As a result, the N−1 viable cell density plateaued at 20.4×10⁶ viable cells/mL when the effective cell bleed from the overflow stream was equivalent to the growth rate. The N−1 bioreactor reached steady state conditions at an average viable cell density of 20.4×10⁶ viable cells/mL, hydrocyclone operation time of 23 min/day, perfusion rate of 1.05 reactor volumes per day (RV/day), and approximated effective cell bleed of 0.5 RV/day on Days 18-23. Following the first steady state, the perfusion rate was increased, and a second steady state was achieved in the N−1 bioreactor at an average viable cell density of 16.5×10⁶ viable cells/mL, hydrocyclone operation time of 30 min/day, perfusion rate of 1.4 RV/day, a CSPR of 80 pL/cell/day, and an approximated effective cell bleed of 0.7 RV/day on Days 27-33.

On Day 4, the overflow stream began to supply cells to three different N stage production CSTRs to simulate N−1 to N volume ratios of 1:5, 1:10, and 1:20. The viable cell density and viability profiles for the production CSTRs can be found in FIGS. 4b, 4c, and 4d.

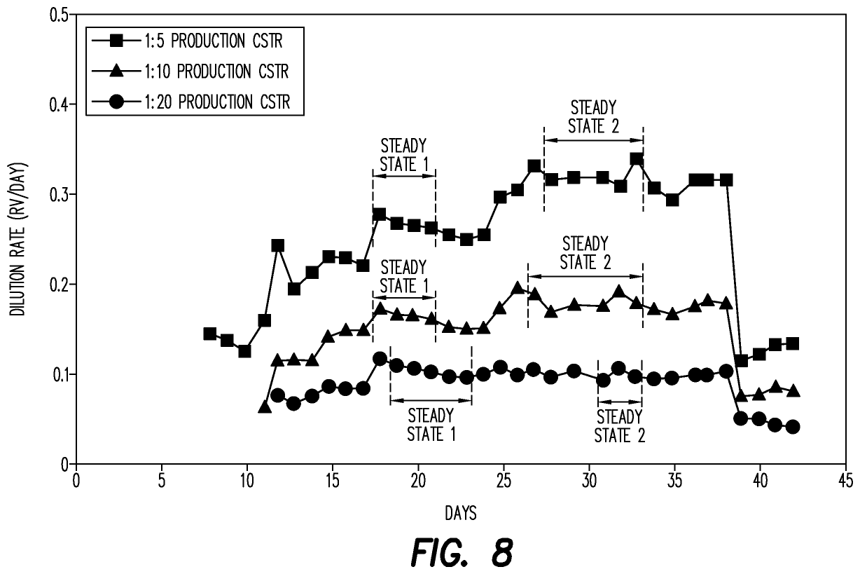

In this case, the cell addition started approximately 16 hours after perfusion so the automated operations could be closely monitored. Whole cell harvest collection began once the bioreactor working volume accumulated to 1.4 L on Days 7, 10, and 11 for the CSTRs simulating 1:5, 1:10, and 1:20 volume ratios, respectively. The CSTR cultures were initially in a highly prolific state with the corresponding tendency to produce high levels of lactic acid; therefore, HIPDOG control, as described earlier, was implemented to maintain lactic acid concentrations at non-inhibitory levels from days 10-18 for the 1:5 volume ratio CSTR, days 11-18 for the 1:10 volume ratio CSTR, and day 13-18 for the 1:20 volume ratio CSTR. Residual glucose and lactate profiles for the production CSTRs are shown in FIGS. 5b, 5c, and 5d. Because the dilution rates were low (less than 0.3 RV/day as shown in FIG. 8) for the CSTRs, sodium/potassium carbonate addition was not required to maintain a residual level of lactate. As the growth rate slowed and the rate of lactic acid production was anticipated to drop, HIPDOG control was discontinued and the glucose feed rate was calculated using historical glucose consumption rates. Following HIPDOG control, the pH was controlled on the low-end by adding a 1M sodium/potassium carbonate (molar ratio as described earlier) and sparging CO₂ on the high-end. Nutrients were semi-continuously supplied directly to the CSTRs using a highly concentrated feed medium to maintain the total residual amino acid levels above approximately 40 mM. Occasional offline UPLC amino acid analysis confirmed that the production CSTRs were not limited for any amino acids. Ammonium levels remained below 6 mM in the 1:5 production CSTR at the steady state timepoints. The 1:10 and 1:20 production CSTRs (operating at low dilution rates) reached slightly higher ammonium levels during Steady State 2 at 7.5 mM and 9.6 mM, respectively. Osmolality was maintained between 320 and 380 mOsm/kg. At steady state timepoints, the production CSTRs maintained cell viabilities of 93-99% (FIGS. 4b, 4c, and 4d) while the N−1 perfusion sustained a cell viability of 91-94% (FIG. 4a). The lower viability in the N−1 bioreactor is likely due to the high shear environment created by the hydrocyclone.

The linked bioreactor systems reached steady state or near steady state conditions for most parameters including viable cell density, bioreactor volume flows, metabolite concentrations, and volumetric productivities. A summary of the steady states is shown in Table 4.

TABLE 4
Summary of linked bioreactor steady states

| | | | | | | Volumetric |
|---|---|---|---|---|---|---|
| | | | | | Steady state | productivity |
| | | | | Average | volumetric | (g/L/day) |
| | | | | perfusion | productivity | calculated by |
| | | | Average | or | (g/L/day) | multiplying |
| | | | VCD | dilution | calculated by | bioreactor titer |
| | Steady | | (×10⁶ | rate | linear | by dilution or |
| Bioreactor | state | Days | cells/mL) | (RV/day) | regression | perfusion rate |
| N-1 perfusion | 1 | 18-23 | 20.4 | 1.05 | 0.54 | 0.51 |
| (operating | 2 | 27-33 | 16.5 | 1.40 | 0.36 | 0.35 |
| independently) | | | | | | |
| 1:5 Simulated | 1 | 18-21 | 19.0 | 0.27 | 0.93 | 0.84 |
| volume ratio | 2 | 28-33 | 15.0 | 0.32 | 0.55 | 0.58 |
| CSTR | | | | | | |
| 1:10 Simulated | 1 | 18-21 | 23.5 | 0.17 | 0.96 | 0.68 |
| volume ratio | 2 | 27-33 | 14.9 | 0.18 | 0.54 | 0.63 |
| CSTR | | | | | | |
| 1:20 Simulated | 1 | 19-23 | 20.9 | 0.10 | 0.84 | 0.47 |
| volume ratio | 2 | 31-33 | 11.5 | 0.10 | 0.54 | 0.55 |
| CSTR | | | | | | |

The linked bioreactor system had a calculated steady-state volumetric productivity of 0.84-0.96 g/L/day for the first steady state and 0.54-0.55 g/L/day for the second steady state. Due to the moderate genetic instability of this CHO cell line, the second steady state productivities are likely slight underestimates. With only minimal process development, at Steady State 1 this continuous linked bioreactor system is 1.8-2 times more productive than the optimized 12-day fed-batch process which has a cumulative volumetric productivity of 0.48 g/L/day, and is also significantly more productive than the N−1 perfusion bioreactor operating independently (0.54 g/L/day). Finally, the results of the linked system using the hydrocyclone compare very favorably with the productivity previously obtained for the same cell line, media, and environmental parameters but using a membrane as the cell retention device. With the Steady State 1 operating conditions, a single hydrocyclone of the design used in the experiments presented here could operate as the cell retention device for a 500-L N−1 perfusion bioreactor with the overflow stream feeding a highly productive production bioreactor between 2,500-10,000 liters without concerns of fouling. Such a linked bioreactor process could potentially operate indefinitely at a highly productive steady state by occasionally (depending upon the genetic stability of the cell line) re-starting the N−1 bioreactor with cells of a lower generational number.

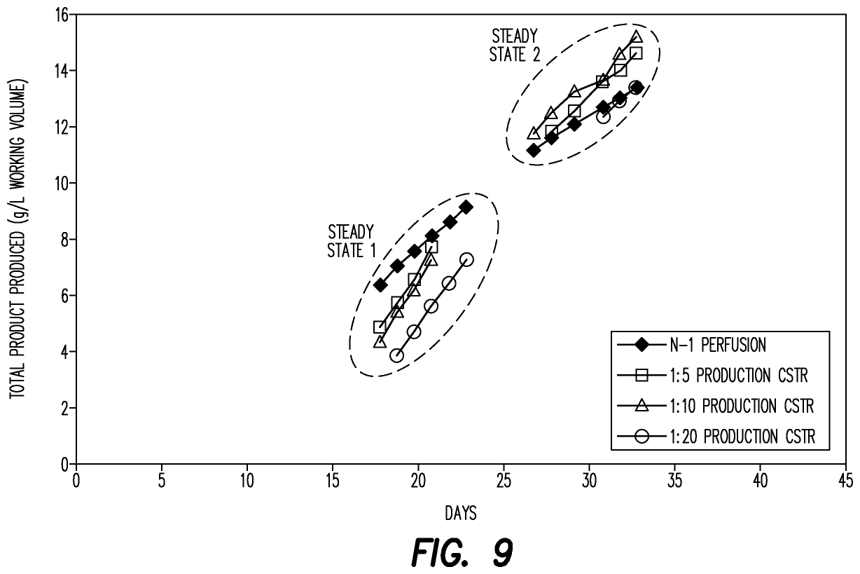

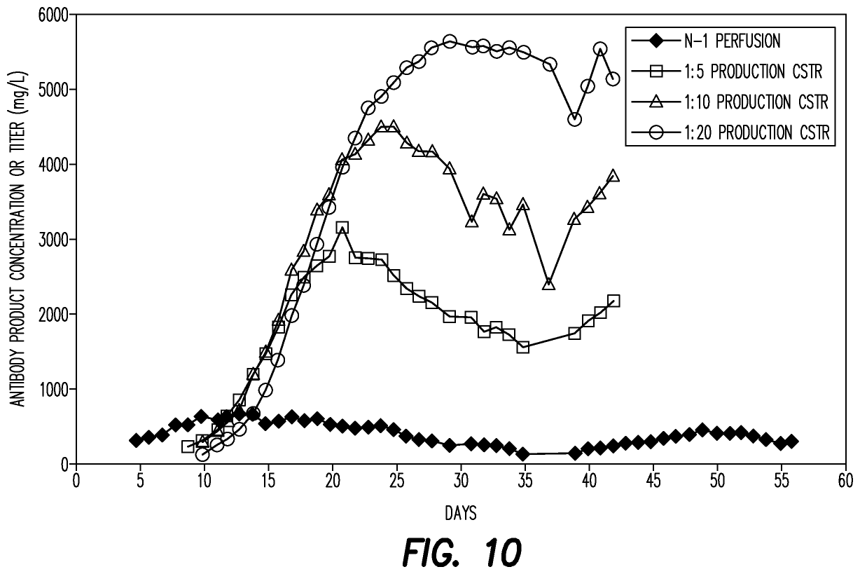

The steady state volumetric productivities were calculated using two methods. In the first and most accurate method, a material balance was performed to determine the rate of antibody product production in the linked bioreactor system. The material balance considered product concentration and volume entering the production CSTR from the N−1 bioreactor, product concentration in the production bioreactor and its rate of removal (dilution rate), and the rate of accumulation of product in the bioreactor. This calculation yielded the total mass of product produced in the linked bioreactor system which was plotted against time as shown in FIG. 9. At time points when the dilution rate, cell density, and metabolites were reasonably constant, a linear regression was used to determine the volumetric productivity in mass of product per volume per time. In the second method product concentration or titer in the bioreactor (FIG. 10) is multiplied by the dilution rate to yield the steady-state volumetric productivity of the system. However, this method would only be accurate if the product concentration was constant and the low dilution rates in the production CSTRs significantly delayed this response.

The use of medium and concentrated feeds in this linked bioreactor system is highly efficient and the required volumes would be reasonable at the manufacturing scale as shown in Table 5. When linked to a CSTR production bioreactor, the N−1 perfusion bioreactor does not generate any waste streams because the entire overflow stream is used to provide cells to the CSTR production bioreactors. The linked bioreactor system would require a total of 0.10-0.32 RV/day (based on the volume of the production reactor) of perfusion medium and concentrated feed in comparison to the 1.05-1.40 RV/day the N−1 perfusion bioreactor requires as a standalone unit operation. Additionally, this linked bioreactor system is also highly efficient in its media usage with 1.72-8.40 grams of product produced per liter of media in comparison to 0.26-0.54 g/L for the independently operating N−1 perfusion and 5.76 g/L for the optimized fed batch process. While we believe the comparisons of the linked bioreactor system to the standalone perfusion bioreactor from these experiments are useful, it is understood that the perfusion bioreactor conditions were not optimized for standalone operation. The growth rate and therefore the required cell bleed rate used in the perfusion bioreactor from these experiments are significantly higher than conditions that would normally be used in a standalone perfusion bioreactor. This is due to the fact that in the case of the linked bioreactor system, the function of the perfusion bioreactor is to deliver a significant number of viable cells in a high growth rate state on a continuous basis to the production bioreactor (CSTR).

TABLE 5
Summary of Media Requirements

| | | | Average | | Mass of |
|---|---|---|---|---|---|
| | | Volume of | volume of | | product |
| | | culture | concen- | Perfusion | per |
| | | received | trated | of | volume |
| | | from N-1 | nutrient | dilution | of liquid |
| | Steady | perfusion | feeds | rate | used |
| Bioreactor | state | (RV/day) | (RV/day)¹ | (RV/day) | (g/L) |
| N-1 perfusion | 1 | N/A | N/A | 1.05 | 0.51 |
| (operating | 2 | N/A | N/A | 1.40 | 0.26 |
| independently) | | | | | |
| 1:5 Simulated | 1 | 0.21 | 0.05 | 0.27 | 3.44 |
| volume ratio | 2 | 0.28 | 0.03 | 0.32 | 1.72 |
| CSTR | | | | | |
| 1:10 Simulated | 1 | 0.11 | 0.06 | 0.17 | 5.65 |
| volume ratio | 2 | 0.14 | 0.04 | 0.18 | 3.00 |
| CSTR | | | | | |
| 1:20 Simulated | 1 | 0.05 | 0.05 | 0.10 | 8.40 |
| volume ratio | 2 | 0.07 | 0.02 | 0.10 | 5.40 |
| CSTR | | | | | |

¹In addition to concentrated nutrient feeds, small volumes 1M sodium/potassium carbonate and 1% simethicone were added to the bioreactors to control pH and foaming, respectively.

On Day 34, there was a perturbation in the N−1 perfusion steady state conditions when the bioreactor volume dropped to approximately 75% of the volume setpoint (volume decreased 25%), likely due to a malfunctioning media pump. During this low volume period, the top impeller was partially exposed and potentially caused excess shear at the liquid/air interface. In an initial attempt to recover the culture, the working volume was restored by delivering additional perfusion media and the perfusion rate was maintained at 1.40 RV/day until Day 38. However, lactate spiked to inhibitory levels as high as 3.2 g/L while the viable cell density and viability reached as low as 4.3×10⁶ viable cells/mL and 86%, respectively. On Day 38, a different approach was taken to recover the culture; the perfusion rate was dropped to 0.4 RV/day to force the culture to consume some of the lactic acid and reduce the cell bleed from the hydrocyclone overflow stream. When the growth rate and viability improved, the overall perfusion rate was slowly increased while maintaining a CSPR of 50 pL/cell/day. On Day 49, the culture had fully recovered to the conditions achieved in the first steady state and conditions maintained reasonably stable until the end of the experiment on Day 56. In retrospect, using a perfusion medium with a lower glucose concentration would have provided more flexibility to provide lactate control and expedite culture recovery.

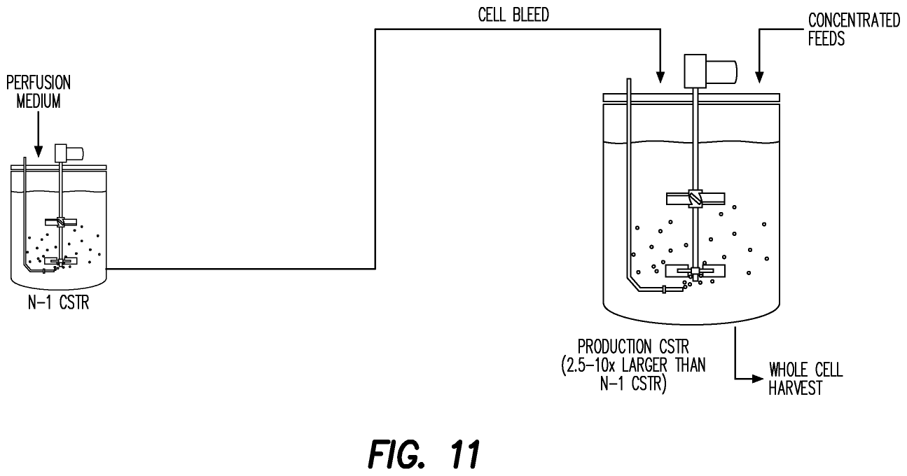

To demonstrate the value of the N−1 perfusion to production CSTR linked bioreactor system using a hydrocyclone, a simpler alternative not requiring perfusion was also evaluated. At the first and most productive steady state in the experiment using the hydrocyclone, the N−1 perfusion maintained a moderate average viable cell density of 20.4×10⁶ viable cells/mL and the hydrocyclone overflow stream that supplied cells to the production CSTRs had an average viable cell density of 10.5×10⁶ cells/mL. This steady state condition had a perfusion rate of 1.05 RV/day, effective cell bleed of 0.54 RV/day, and a CSPR of 50 pL/cell/day. It was contemplated that the N−1 bioreactor could be operated as a CSTR to add the same volume and quantity of cells to the production CSTRs if the volume of the N−1 bioreactor was doubled and culture conditions maintaining a high growth rate were utilized. A diagram of the cascading CSTR process is shown in FIG. 11.

An experiment was performed to compare these cascading CSTRs to the results achieved with the linked bioreactors using the hydrocyclone. Because it was predicted than an N−1 CSTR would not achieve as high cell densities as the N−1 perfusion bioreactor using the hydrocyclone, in this experiment, the simulated volume ratios of the N−1 bioreactor to the production CSTRs were halved. Therefore, the simulated volume ratios between the N−1 CSTR and the N-stage production CSTRs were 1:2.5, 1:5, and 1:10. A summary of operating parameters are shown in Table 6.

TABLE 6
Operating parameters for the N-1 CSTR and the Production CSTRs

| | | Production CSTR |
|---|---|---|
| Parameter | N-1 CSTR | Bioreactors |
| Inoculation Density (viable | 0.8 × 10⁶ | 0 |
| cells/mL) | | |
| Temperature (° C.) | 36.5 | 36.5 |
| pH setpoint during | 7.08 ± 0.05 (controlled by | 7.08 ± 0.05 (controlled by |
| HIPDOG | 1M sodium/potassium | 1M sodium/potassium |
| | carbonate on the low end | carbonate on the low end |
| | and 500 g/L glucose on the | and 500 g/L glucose on the |
| | high end) | high end) |
| pH setpoint post HIPDOG | N/A | 7.05 ± 0.10 (controlled by |
| | | 1M sodium/potassium |
| | | carbonate on the low end |
| | | and CO₂ on the high end) |
| Dissolved oxygen setpoint | 40 | 40 |
| (% air saturation) | | |
| Dissolved CO2 (% of | 4-10 | 4-10 |
| saturation) | | |
| Working volume (L) | 1.4 | 1.4 |
| Agitation | Single 4.5 cm diameter | Single 4.5 cm diameter |
| | Rushton impeller at 80 W/m³ | Rushton impeller at 80 W/m³ |
| | power/unit volume | power/unit volume |

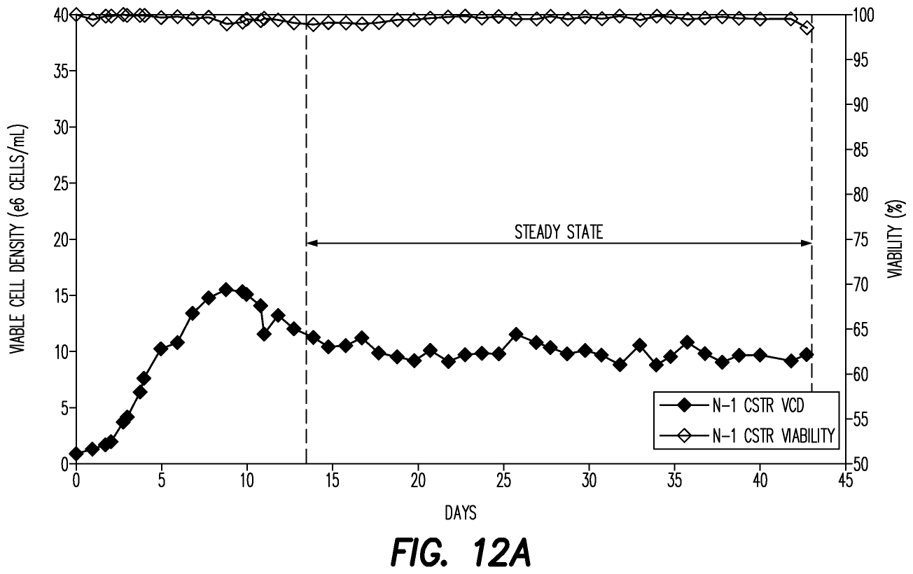

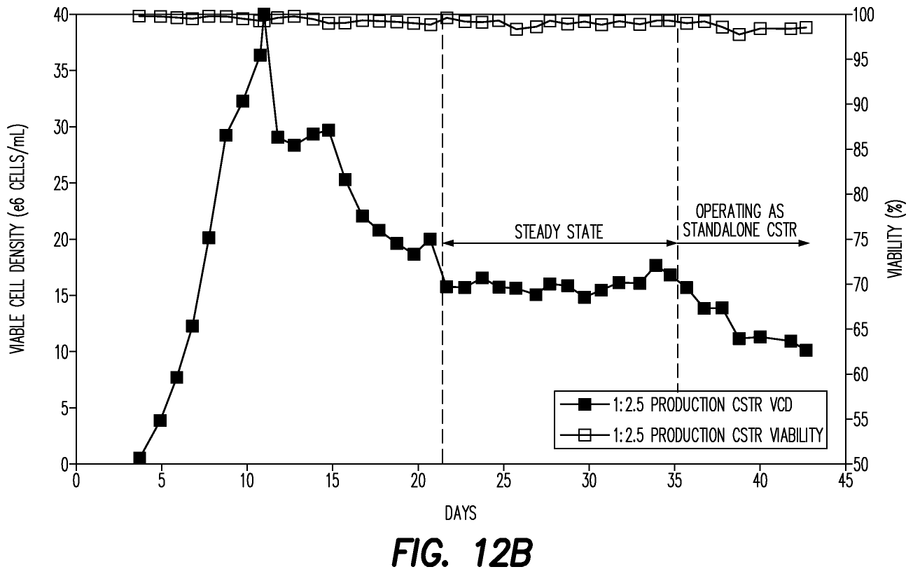

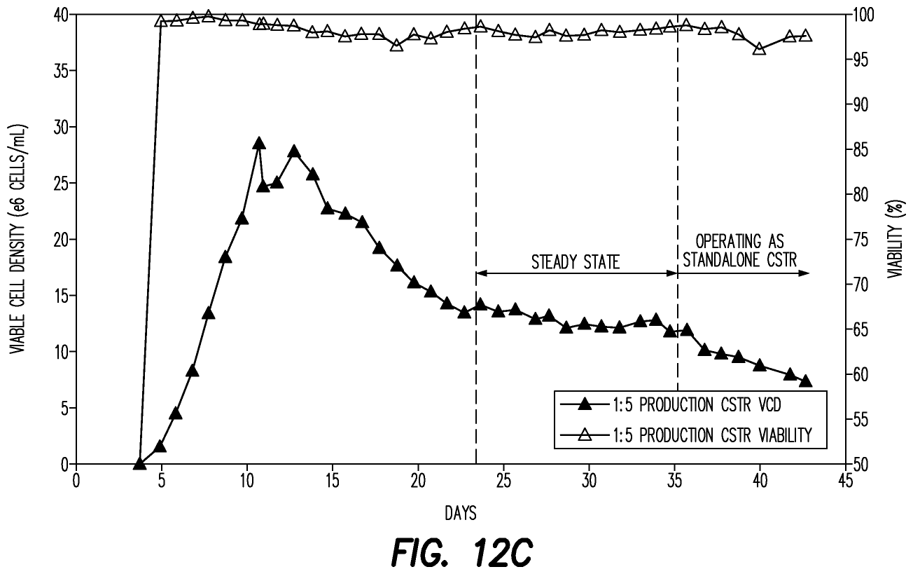

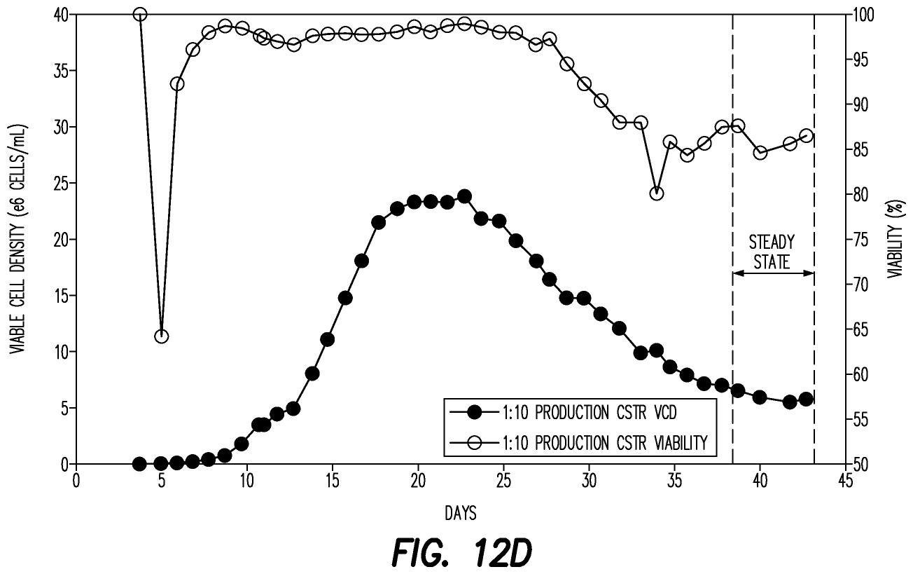

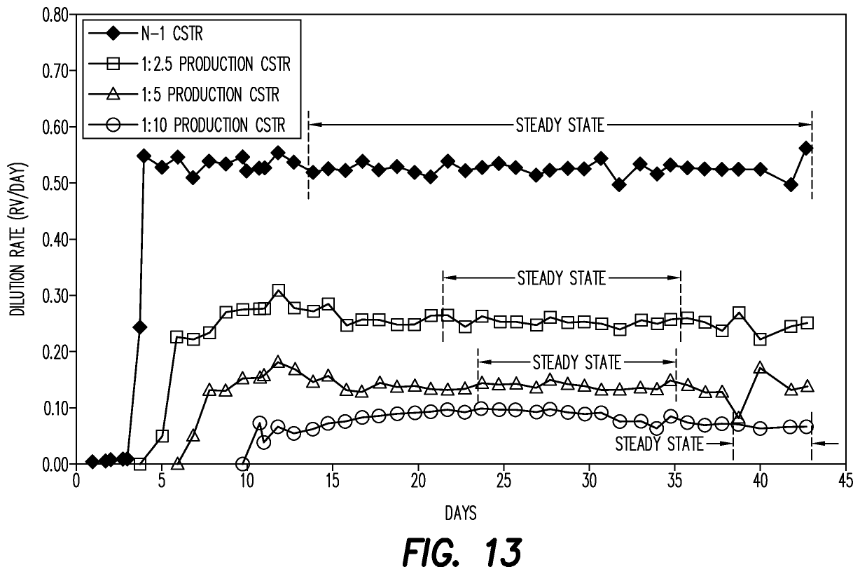

The N−1 CSTR was a 3-L glass vessel (Applikon, Scheidam, Netherlands) operating at a 1.4-L working volume. The bioreactor was maintained at constant volume by a ChemTec pump (Parker Domnik Hunter, Oxnard, CA) that removed whole cell culture from the N−1 bioreactor in response to an increase in bioreactor weight. The N−1 CSTR initially utilized a 7×1 mm drilled-hole sparger to supply oxygen to the culture. Once the oxygen demand reached high levels, a 15 m sintered steel sparger was used to supply oxygen to the culture while oxygen was sparged through the 7×1 mm drilled-hole sparger to supply oxygen to the culture and strip CO₂. In order to maintain the dissolved CO₂ conditions listed in Table 6, occasional manual adjustments were made to the oxygen flow rate through the drilled-hole sparger based on offline CO₂ measurements. The N−1 CSTR was inoculated at 0.8×10⁶ cells/mL with the same CHO cell line and basal medium (Table 2) used in the linked bioreactor experiment with the hydrocyclone. The viable cell density and cell viability profile can be found in FIG. 12a. During the first two days of the N−1 process, there was a significant offset with the temperature probe that caused the bioreactor to be controlled at approximately 3° C. lower than the temperature setpoint, resulting in a lag in growth. The temperature was corrected on Day 2 and the culture quickly returned to exponential growth. Dilution began on Day 3 at a dilution rate of 0.25 RV/day by adding the same medium as used in the hydrocyclone experiments in Table 2. The dilution rate was increased to 0.54 RV/day on Day 4 and held constant for the remainder of the experiment as shown in FIG. 13. The dilution medium was added semi-continuously to the N−1 bioreactor in equivalent doses every 15 minutes.

The production CSTRs were 3-L glass vessels (Applikon, Scheidam, Netherlands) operating at a 1.4-L working volume. The production CSTRs utilized the same dual sparging strategy as described for the N−1 CSTR. Like the linked N−1 perfusion to CSTR bioreactor system with the hydrocyclone, it is intended that in a manufacturing setting, the entire cell bleed from the N−1 CSTR would feed a production CSTR between 2.5 to 10 times larger by volume. However, in this experimental setup, the volume ratios were simulated by adding the appropriate volume of cell bleed to the production CSTRs while the excess cell bleed volume was directed to waste. The N−1 cell bleed was added semi-continuously (as described previously) to the production bioreactors in equivalent doses every 15 minutes. The production CSTRs initially contained only basal medium at 70% of the final working volume. Once the volume of the production CSTR accumulated to the final working volume of 1.4 L, the volume was maintained constant by a ChemTec pump (Parker Domnik Hunter, Oxnard, CA) that removed whole cell harvest from the bioreactor. Viable cell density and cell viability data for the production CSTRs are shown in FIGS. 12b, 12c, and 12d.

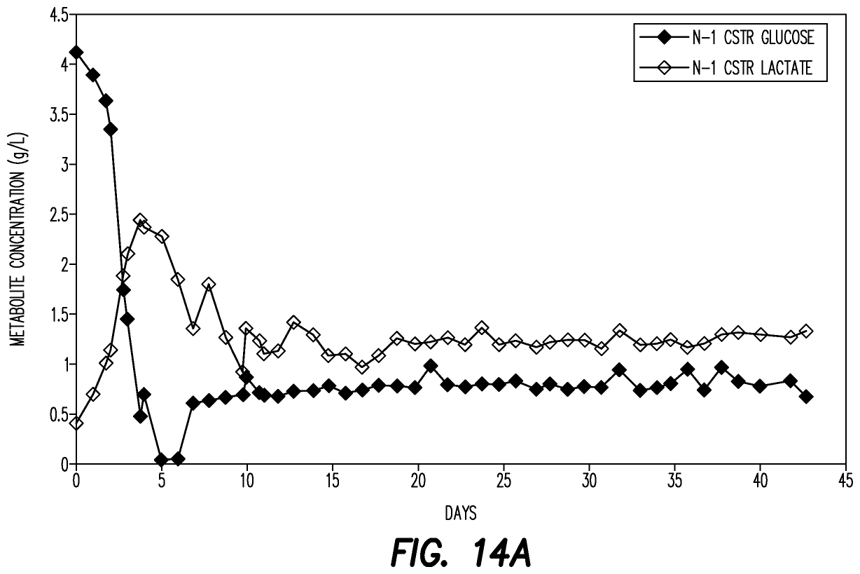

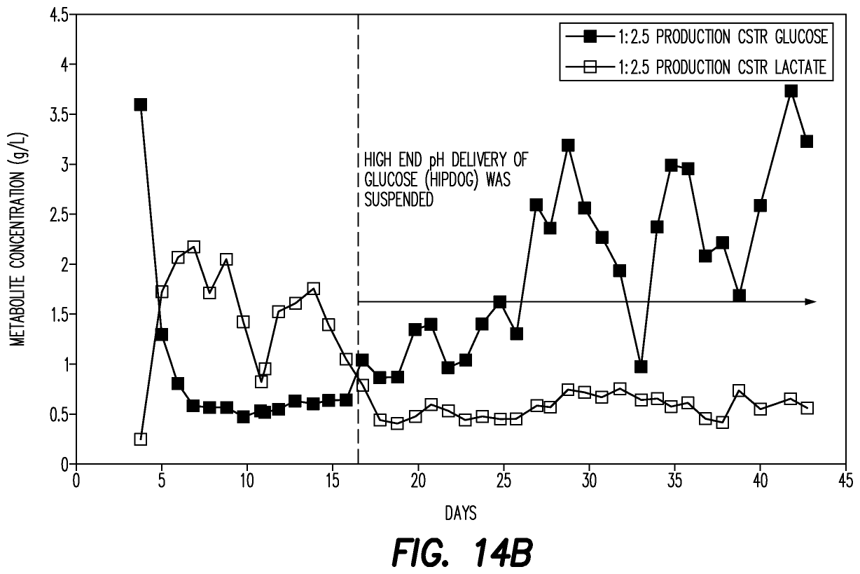

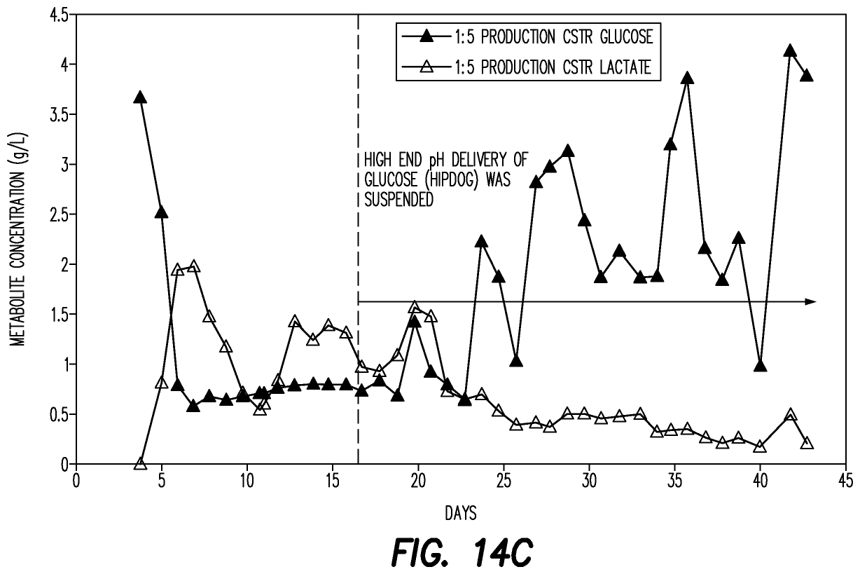

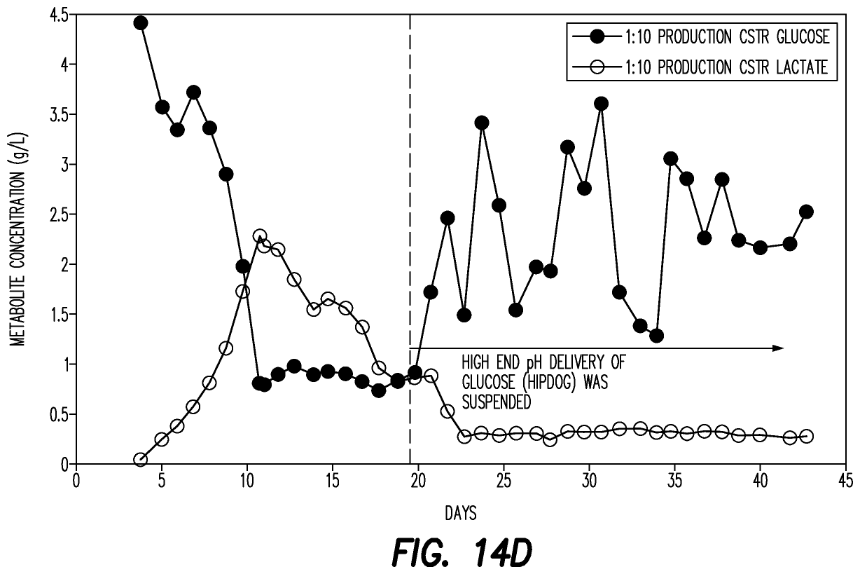

Lactate accumulation was controlled using HIPDOG control as previously described. The N−1 CSTR was operated at a relatively high dilution rate and therefore maintained conditions conducive to a high cellular growth rate. In this highly proliferative state, the N−1 culture was prone to producing high levels of lactic acid and HIPDOG control was used starting on Day 7 for the duration of the experiment to maintain lactate at non-inhibitory levels. The production CSTRs were initially at a high growth rate with the corresponding propensity to produce lactic acid. HIPDOG control was used to control lactate accumulation in the production CSTRs on Days 5-17 for the 1:2.5 simulated volume ratio, Days 6-17 for the 1:5 simulated volume ratio, and Days 11-20 for the simulated 1:10 volume ratio. Following HIPDOG, the glucose feed rate was calculated using historical glucose consumption rates and the bioreactors were generally not limited with respect to glucose availability. Residual glucose and lactate concentrations for the N−1 CSTR and the production CSTRs is shown in FIGS. 14a, 14b, 14c, and 14d. After HIPDOG control was discontinued, pH was controlled on the low-end by adding a 1M sodium/potassium (molar ratio described earlier) solution and controlled on the high-end by sparging CO₂. A highly concentrated feed medium semi-continuously supplied additional nutrients directly to the production CSTRs. The nutrient feed rates were adjusted to maintain the residual amino acid concentration above 40 mM. Occasional offline UPLC analysis confirmed that the N−1 and production CSTRs were not limited for any amino acids. While lactate accumulation was well controlled (FIGS. 14a, 14b, 14c, and 14d), ammonium accumulated to a maximum of 7.5 5.5, 8.9, and 15.5 mM during the steady state conditions for the N−1, 1:2.5, 1:5, and 1:10 volume ratios CSTRs, respectively. Osmolality was well-maintained between 280 and 370 mOsm/kg.

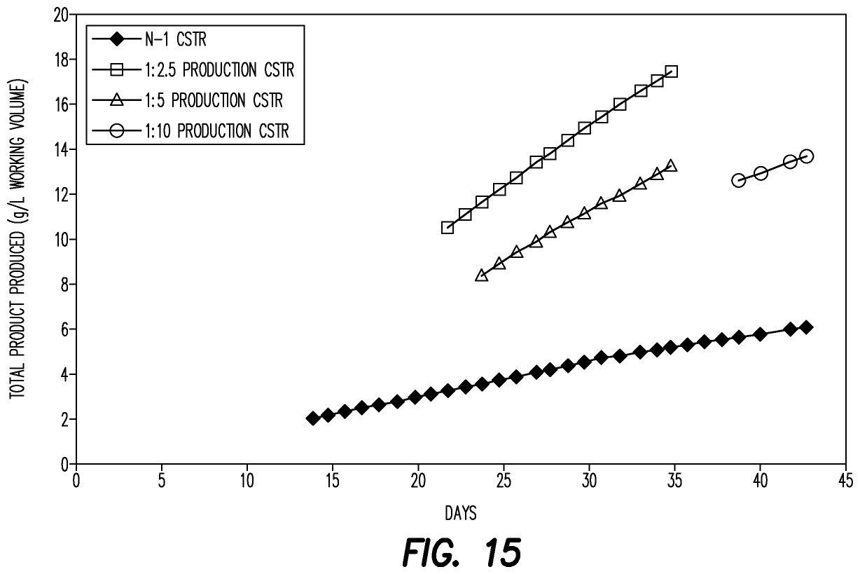

The N−1 CSTR reached a steady state on day 14 at a viable cell density of 10.0×106 viable cells/mL, dilution rate of 0.53 RV/day, and a cell specific dilution rate of 50 pL/cell/day. The production CSTRs reached steady state conditions for most parameters including viable cell density, dilution rate, metabolite concentration, and volumetric productivities at various time points as indicated on FIGS. 12b, 12c, and 12d. Additionally, a summary of the steady state conditions is shown in Table 7. At the steady state timepoints, the N−1 CSTR and the production CSTRs simulating 1:2.5 and 1:5 volume ratios sustained cell viabilities exceeding 97% (FIGS. 12a, 12b, and 12c). The viability in the production CSTR simulating a 1:10 volume ratio had a lower steady state viability of 83-87% (FIG. 12d), potentially due to the accumulation of certain inhibitory metabolites at this low dilution rate seen previously for this cell line. The steady state volumetric productivities for the cascading CSTRs were calculated using the same methods as described for the N−1 perfusion linked to a production CSTR using the hydrocyclone (FIG. 15).

TABLE 7
Summary of Cascading CSTRs Steady States

| | | | | | Volumetric |
|---|---|---|---|---|---|
| | | | | Steady state | productivity |
| | | | | volumetric | (g/L/day) |
| | | | Average | productivity | calculated by |
| | | Average | dilution | (g/L/day) | multiplying |
| | | VCD | rate | calculated by | bioreactor |
| | | (×10⁶ | (RV/ | linear | titer by |
| Bioreactor | Days | cells/mL) | day) | regression | dilution rate |
| N-1 CSTR | 14-43 | 10.0 | 0.53 | 0.14 | 0.11 |
| (operating | | | | | |
| independently) | | | | | |
| 1:2.5 Simulated | 22-35 | 16.1 | 0.25 | 0.53 | 0.51 |
| volume ratio | | | | | |
| CSTR | | | | | |
| 1:5 Simulated | 24-35 | 12.9 | 0.14 | 0.43 | 0.44 |
| volume ratio | | | | | |
| CSTR | | | | | |
| 1:10 Simulated | 39-43 | 6.0 | 0.07 | 0.27 | 0.33 |
| volume ratio | | | | | |
| CSTR | | | | | |

Although the N−1 perfusion linked to production CSTR with the hydrocyclone requires a more complex equipment setup, it is between 1.8 and 3.1 times more productive than the cascading CSTR systems.

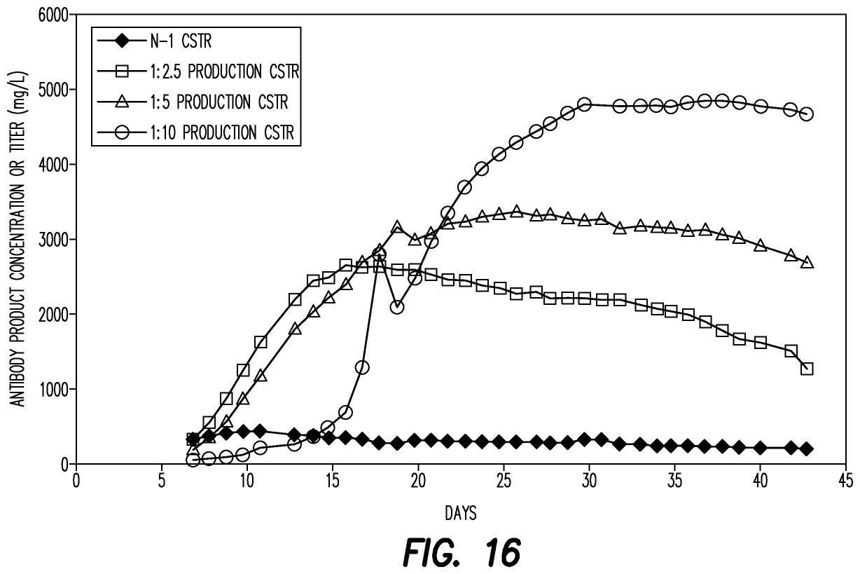

After the 1:2.5 volume ratio and 1:5 volume ratio cascading CSTR systems maintained steady state conditions for several days, these two production CSTRs were operated in a standalone mode to determine the value of the cascading operation. On Days 35-43, the cell bleed addition from the N−1 CSTR to the production CSTRs was stopped. To maintain equivalent dilution rates, fresh perfusion medium was added to the production CSTRs in place of the N−1 cell bleed. Once the addition of fresh cells from the N−1 CSTR had stopped, a drop VCD was observed. Correspondingly, the bioreactor titer (FIG. 16) dropped which indicates a drop in volumetric productivity.

These results demonstrate that the exemplary continuous linked bioreactor system using a hydrocyclone as a cell retention device is highly productive and media-efficient. The exemplary systems and methods described herein have the potential to reduce manufacturing costs and increase volumetric productivity in existing manufacturing facilities while eliminating concerns of membrane fouling.

In a manufacturing setting, a single hydrocyclone of the design disclosed herein could operate as the cell retention device on a highly proliferative 500-L N−1 perfusion while supplying cells to a 2,500-10,000-L production CSTR. This system would operate at a steady state volumetric productivity as high as 0.96 g/L/day, require only 0.17 RV/day of media, and deliver a continuous stream of antibody product at 5.7 grams/L. These volumetric productivities are comparable to a linked bioreactor system using a membrane cell retention device and up to twice as productive as the optimized fed-batch process. Perfusion with the hydrocyclone would have significant advantages if applied to large scale bioreactors for which conventional membrane perfusion methods may be impractical. Although we found that the hydrocyclone had operational limitations for other modes of perfusion, it was successfully used for a highly productive linked bioreactor system that could be adapted for a legacy manufacturing facility without extraordinary facility modification, enabling the economic benefits of a continuous or semi-continuous downstream purification process.

In the preceding specification, various embodiments have been described with reference to the examples. It will, however, be evident that various modifications and changes may be made thereto, and additional embodiments may be implemented, without departing from the broader scope of the exemplary embodiments as set forth in the claims that follow. The specification and drawings are accordingly to be regarded in an illustrative rather than restrictive sense.

1. A system for continuous production of at least one biologic molecule of interest, the system comprising:
(i) at least one culture bioreactor;
(ii) at least one hydrocyclone; and
(iii) at least one production bioreactor;
wherein the culture bioreactor and the production bioreactor are linked by the hydrocyclone that serves as a cell retention device for the culture bioreactor, wherein the hydrocyclone produces an underflow stream containing concentrated mammalian cell culture and a partially cell-free overflow stream, and wherein the underflow stream containing concentrated mammalian cell culture is returned to the culture bioreactor and the partially cell-free overflow stream is directed to the production bioreactor.

2. A system for continuous production of at least one biological molecule of interest, the system comprising:
(i) at least one culture bioreactor;
(ii) at least one hydrocyclone;
(iii) at least one production bioreactor; and
(iv) at least one downstream component;
wherein the culture bioreactor and the production bioreactor are linked by the hydrocyclone that serves as a cell retention device for the culture bioreactor, wherein the hydrocyclone produces an underflow stream containing concentrated mammalian cell culture and a partially cell-free overflow stream, and wherein the underflow stream containing concentrated mammalian cell culture is returned to the culture bioreactor and the partially cell-free overflow stream is directed to the production bioreactor.

3. The system of claim 1, wherein the at least one culture bioreactor is a N−1 bioreactor or a N−1 perfusion bioreactor.

4. The system of claim 1, wherein the at least one production bioreactor is an N bioreactor or a continuously stirred tank reactor (CSTR) production bioreactor.

5. The system of claim 1, wherein the system can operate in a highly productive steady state for more than six weeks.

6. The system of claim 1, wherein about 85 to about 95% of the cells that enter the hydrocyclone are retained and returned to the culture bioreactor via the underflow stream.

7. The system of claim 1, wherein the hydrocyclone has the following dimensions: an overflow diameter in the range of about 1 to about 3 mm, an underflow diameter in the range about 1 to about 4 mm, a chamber diameter in the range about 5 to about 15 mm, a conical section length in the range about 50 to about 170 mm, a cylindrical section length in the range of about 2 to about 20 mm, a vortex finder length in the range of about 1 to about 4 mm, and an underflow outlet length in the range of about 0 to about 40 mm.

8. The system of claim 1, wherein the system has a working volume of about 0.5 to about 25,000 L.

9. The system of claim 1, wherein the system is a manufacturing- or production-scale system that has a working volume of about 500 to about 25,000 L.

10. The system of claim 1, wherein the culture bioreactor has a working volume in the range of about 0.02 L to about 5,000 L.

11. The system of claim 1, wherein the production bioreactor has a working volume in the range of about 0.4 to about 25,000 L.

12. The system of claim 1, wherein the ratio of the working volume of the culture bioreactor to the working volume of the production bioreactor is in the range of about 1:5 to about 1:20.

13. The system of claim 2, wherein the at least one downstream component is selected from precipitation components, purification components, finishing components, packaging components or combinations thereof.

14. The system of claim 2, wherein the at least one downstream component is not linked to the system.

15. The system of claim 2, wherein the at least one downstream component is linked to the system or is linked to the other elements of the continuous system.

16. The system of claim 1, wherein the mammalian cell culture comprises mammalian cells selected from NSO, Sp2/0-Ag14, BHK21, BHK TK⁻, HaK, 2254-62.2 (BHK-21 derivative), CHO, CHO wild type, CHO-DUKX, CHO-DUKX B11, CHO-DG44, CHO Pro-5, CHO-S, Lec13, V79, HEK 293, COS-7, HuNS1, Per.C6, CHO-K1, CHO-K1/SF, CHO-K1 GS, CHOZN GS. In one embodiment, the mammalian cells are selected from the group consisting of: CHO cells, HEK-293 cells, VERO cells, NSO cells, PER.C6 cells, Sp2/0 cells, BHK cells, MDCK cells, MDBK cells, and COS cells.

17. A method for producing a biologic molecule of interest, the method comprising:
(i) culturing a plurality of host cells capable of producing a biologic molecule of interest in at least one culture bioreactor;
(ii) inoculating at least one production bioreactor with cells obtained from step (i); and
(iii) culturing the cells in the production bioreactor;
wherein the culture bioreactor and the production bioreactor are linked by at least one hydrocyclone that serves as a cell retention device for the at least one culture bioreactor; and
wherein the host cells comprise mammalian cells.

18. A method for producing a biologic molecule of interest, the method comprising:
(i) culturing a plurality of host cells in at least one culture bioreactor;
(ii) inoculating at least one production bioreactor with cells obtained from step (i); and
(iii) culturing the cells in the production bioreactor under conditions that allow production of the biologic molecule of interest;
wherein the culture bioreactor and the production bioreactor are linked by at least one hydrocyclone that serves as a cell retention device for the culture bioreactor,
wherein the host cells comprise mammalian cells, and
wherein the hydrocyclone produces an underflow stream containing concentrated mammalian cell culture and a partially cell-free overflow stream, and wherein the underflow stream containing concentrated mammalian cell culture is returned to the culture bioreactor and the partially cell-free overflow stream is directed to the production bioreactor.

19. The method of claim 18, wherein the inoculation in step (ii) is by transferring cells from the culture bioreactor to the production bioreactor.

20. The method of claim 19, wherein the transferring of the cells from the culture bioreactor to the production bioreactor is by cell bleed in continuous or semi-continuous modes.

21. The method of claim 20, wherein the cell bleed is in semi-continuous mode.

22. The method of claim 18, wherein the systems for use in the methods do not comprise a membrane cell retention device.

23. The method of claim 18, wherein the at least one production bioreactor receives a host cell transfer from the culture bioreactor by way of the hydrocyclone overflow once at every period of time from 2 minutes to 24 hours or at any interval therebetween.

24. The method of claim 18, wherein the hydrocyclone is continuously operating.

25. The method of claim 18, wherein the hydrocyclone is continuously receiving culture from the culture bioreactor.

26. The method of claim 18, wherein the mammalian cell culture comprises mammalian cells selected from NSO, Sp2/0-Ag14, BHK21, BHK TK⁻, HaK, 2254-62.2 (BHK-21 derivative), CHO, CHO wild type, CHO-DUKX, CHO-DUKX B11, CHO-DG44, CHO Pro-5, CHO-S, Lec13, V79, HEK 293, COS-7, HuNS1, Per.C6, CHO-K1, CHO-K1/SF, CHO-K1 GS, CHOZN GS. In one embodiment, the mammalian cells are selected from the group consisting of: CHO cells, HEK-293 cells, VERO cells, NSO cells, PER.C6 cells, Sp2/0 cells, BHK cells, MDCK cells, MDBK cells, and COS cells.
